(19) United States
(12) Patent Application Publication
(10) Pub. No.: US 2026/0021096 A1
(43) Pub. Date: Jan. 22, 2026

(54) TREATMENT OF HIDRADENITIS SUPPURATIVA USING JAK INHIBITORS

(71) Applicant: Incyte Corporation, Wilmington, DE (US)

(72) Inventors: Michael D. HOWELL, Kennett Square, PA (US); Paul SMITH, Wilmington, DE (US)

(73) Assignee: Incyte Corporation, Wilmington, DE (US)

(21) Appl. No.: 19/059,472

(22) Filed: Feb. 21, 2025

Related U.S. Application Data
(63) Continuation of application No. 17/720,077, filed on Apr. 13, 2022, now Pat. No. 12,280,054.
Application No. 17/720,077 is a division of application No. 16/370,436, filed on Mar. 29, 2019, now Pat. No. 11,304,949.
(60) Provisional application No. 62/650,600, filed on Mar. 30, 2018.

(51) Int. Cl. A61K 31/519; A61K 31/573; A61K 45/06

(52) U.S. Cl. CPC A61K 31/519; A61K 31/573; A61K 45/06

(57) ABSTRACT

The present application provides methods of treating hidradenitis suppurativa in a patient in need thereof, comprising administering to the patient a therapeutically effective amount of a compound which inhibits JAK1 and/or JAK2, or a pharmaceutically acceptable salt thereof.

The present application claims the benefit of U.S. Provisional Application No. 62/650,600, filed Mar. 30, 2018, which is incorporated herein by reference in its entirety.

### TECHNICAL FIELD

The present application provides methods for the treatment hidradenitis suppurativa (HS) using compounds that modulate the activity of Janus kinase (JAK) 1 and/or 2.

### BACKGROUND

Protein kinases (PKs) regulate diverse biological processes including cell growth, survival, differentiation, organ formation, morphogenesis, neovascularization, tissue repair, and regeneration, among others. Protein kinases also play specialized roles in a host of human diseases including cancer. Cytokines, low-molecular weight polypeptides or glycoproteins, regulate many pathways involved in the host inflammatory response to sepsis. Cytokines influence cell differentiation, proliferation and activation, and can modulate both pro-inflammatory and anti-inflammatory responses to allow the host to react appropriately to pathogens. Signaling of a wide range of cytokines involves the Janus kinase family (JAKs) of protein tyrosine kinases and Signal Transducers and Activators of Transcription (STATs). There are four known mammalian JAKs: JAK1 (Janus kinase-1), JAK2, JAK3 (also known as Janus kinase, leukocyte; JAKL; and L-JAK), and TYK2 (protein-tyrosine kinase 2).

Cytokine-stimulated immune and inflammatory responses contribute to pathogenesis of diseases: pathologies such as severe combined immunodeficiency (SCID) arise from suppression of the immune system, while a hyperactive or inappropriate immune/inflammatory response contributes to the pathology of autoimmune diseases (e.g., asthma, systemic lupus erythematosus, thyroiditis, myocarditis), and illnesses such as scleroderma and osteoarthritis (Ortmann, R. A., T. Cheng, et al. (2000) Arthritis Res 2(1): 16-32).

Deficiencies in expression of JAKs are associated with many disease states. For example, Jak1−/− mice are runted at birth, fail to nurse, and die perinatally (Rodig, S. J., M. A. Meraz, et al. (1998) Cell 93(3): 373-83). Jak2−/− mouse embryos are anemic and die around day 12.5 postcoitum due to the absence of definitive erythropoiesis.

The JAK/STAT pathway, and in particular all four JAKs, are believed to play a role in the pathogenesis of asthmatic response, chronic obstructive pulmonary disease, bronchitis, and other related inflammatory diseases of the lower respiratory tract. Multiple cytokines that signal through JAKs have been linked to inflammatory diseases/conditions of the upper respiratory tract, such as those affecting the nose and sinuses (e.g., rhinitis and sinusitis) whether classically allergic reactions or not. The JAK/STAT pathway has also been implicated in inflammatory diseases/conditions of the eye and chronic allergic responses.

Activation of JAK/STAT in cancers may occur by cytokine stimulation (e.g. IL-6 or GM-CSF) or by a reduction in the endogenous suppressors of JAK signaling such as SOCS (suppressor or cytokine signaling) or PIAS (protein inhibitor of activated STAT) (Boudny, V., and Kovarik, J., Neoplasm. 49:349-355, 2002). Activation of STAT signaling, as well as other pathways downstream of JAKs (e.g., Akt), has been correlated with poor prognosis in many cancer types (Bowman, T., et al. Oncogene 19:2474-2488, 2000). Elevated levels of circulating cytokines that signal through JAK/STAT play a causal role in cachexia and/or chronic fatigue. As such, JAK inhibition may be beneficial to cancer patients for reasons that extend beyond potential anti-tumor activity.

JAK2 tyrosine kinase can be beneficial for patients with myeloproliferative disorders, e.g., polycythemia vera (PV), essential thrombocythemia (ET), myeloid metaplasia with myelofibrosis (MMM) (Levin, et al., Cancer Cell, vol. 7, 2005: 387-397). Inhibition of the JAK2V617F kinase decreases proliferation of hematopoietic cells, suggesting JAK2 as a potential target for pharmacologic inhibition in patients with PV, ET, and MMM.

Inhibition of the JAKs may benefit patients suffering from skin immune disorders such as psoriasis, and skin sensitization. The maintenance of psoriasis is believed to depend on a number of inflammatory cytokines in addition to various chemokines and growth factors (JCI, 113:1664-1675), many of which signal through JAKs (Adv Pharmacol. 2000;47:113-74).

Thus, new or improved agents which inhibit kinases such as JAKs are continually needed for developing new and more effective pharmaceuticals that are aimed at augmentation or suppression of the immune and inflammatory pathways, such as the treatment of hidradenitis suppurativa. This application is directed to that need and others.

### SUMMARY

The present application provides methods of treating hidradenitis suppurativa in a patient in need thereof, comprising administering to the patient a therapeutically effective amount of a compound which inhibits JAK1 and/or JAK2, or a pharmaceutically acceptable salt thereof.

In some embodiments, the compound or salt is selective for JAK1 and JAK2, which is selective over JAK3 and TYK2.

In some embodiments, the compound or salt is selective for JAK1 over JAK2, JAK3, and TYK2.

In some embodiments, the compound is ruxolitinib, or a pharmaceutically acceptable salt thereof.

In some embodiments, the compound is ruxolitinib, or a pharmaceutically acceptable salt thereof, wherein one or more hydrogen atoms are replaced by deuterium atoms.

In some embodiments, the salt is ruxolitinib phosphate.

In some embodiments, the compound is {1-{1-[3-fluoro-2-(trifluoromethyl)isonicotinoyl]piperidin-4-yl}-3[4-(7H-pyrrolo[2,3-d]pyrimidin-4-yl)-1H-pyrazol-1-yl]azetidin-3-yl}acetonitrile, or a pharmaceutically acceptable salt thereof.

In some embodiments, the salt is {1-{1-[3-fluoro-2-(trifluoromethyl)isonicotinoyl]piperidin-4-yl}-3[4-(7H-pyrrolo[2,3-d]pyrimidin-4-yl)-1H-pyrazol-1-yl]azetidin-3-yl}acetonitrile adipic acid salt.

In some embodiments, the compound is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide, or a pharmaceutically acceptable salt thereof.

In some embodiments, the salt is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide phosphoric acid salt.

In some embodiments, the compound or salt is administered at a dosage of 15, 30, 60 or 90 mg on a free base basis.

In some embodiments, the compound is ((2R,5S)-5-{2-[(1R)-1-hydroxyethyl]-1H-imidazo[4,5-d]thieno[3,2-b]pyridin-1-yl}tetrahydro-2H-pyran-2-yl)acetonitrile, or a pharmaceutically acceptable salt thereof.

In some embodiments, the compound is ((2R,5S)-5-{2-[(1R)-1-hydroxyethyl]-1H-imidazo[4,5-d]thieno[3,2-b]pyridin-1-yl}tetrahydro-2H-pyran-2-yl)acetonitrile monohydrate.

In some embodiments, the methods further comprise administering an additional therapeutic agent (e.g., an antibiotic, a retinoid, a corticosteroid, an anti-TNF-alpha agent, or an immunosuppressant).

In some embodiments, the administrating of the compound or salt is topical. In some embodiments, the administering of the compound or salt is oral.

In some embodiments, the method results in a 10%, 20%, 30%, 40%, or 50% improvement in HiSCR (Hidradenitis Suppurativa Clinical Response).

The present application also provides a compound which inhibits JAK1 and/or JAK2, or a pharmaceutically acceptable salt thereof, for use in treating hidradenitis suppurativa.

The present application further provides use of a compound which inhibits JAK1 and/or JAK2, or a pharmaceutically acceptable salt thereof, for preparation of a medicament for use in treatment of hidradenitis suppurativa.

### BRIEF DESCRIPTION OF THE DRAWINGS

FIG. 1 illustrates the individual gene expression values (MFI) for JAK1, for each experimental replicate in keratinocytes simulated with TNFα and IFN-γ in the presence/absence of Compounds A-D. Keratinocytes were stimulated with TNFα (25 ng/ml) and IFNγ (25 ng/ml) in the presence/absence of increasing concentrations of JAK inhibitors. Data are presented as JAK1 expression levels for each group.

FIG. 2 illustrates the individual gene expression values (MFI) for JAK2, for each experimental replicate in keratinocytes simulated with TNFα and IFN-γ in the presence/absence of Compounds A-D. Keratinocytes were stimulated with TNFα (25 ng/mL) and IFNγ (25 ng/ml) in the presence/absence of increasing concentrations of JAK inhibitors. Data are presented as JAK2 expression levels for each group.

FIG. 3 illustrates the individual gene expression values (MFI) for IL-1α for each experimental replicate in keratinocytes simulated with TNFα and IFN-γ in the presence/absence of Compounds A-D. Keratinocytes were stimulated with TNFα (25 ng/mL) and IFNγ (25 ng/ml) in the presence/absence of increasing concentrations of JAK inhibitors. Data are presented as IL-1α expression levels for each group.

FIG. 4 illustrates the individual gene expression values (MFI) for IL-6, for each experimental replicate in keratinocytes simulated with TNFα and IFN-γ in the presence/absence of Compounds A-D. Keratinocytes were stimulated with TNFα (25 ng/mL) and IFNγ (25 ng/ml) in the presence/absence of increasing concentrations of JAK inhibitors. Data are presented as IL-6 expression levels for each group.

FIG. 5 illustrates the individual protein concentrations (pg/mL) for IL-1α, for each experimental replicate in keratinocytes simulated with TNFα and IFN-γ in the presence/absence of Compounds A-D. Keratinocytes were stimulated with TNFα (25 ng/ml) and IFNγ (25 ng/mL) in the presence/absence of increasing concentrations of JAK inhibitors. Data are presented as IL-1α concentrations for each group.

FIG. 6 illustrates the individual protein concentrations (pg/mL) for IL-6, for each experimental replicate in keratinocytes simulated with TNFα and IFN-γ in the presence/absence of Compounds A-D. Keratinocytes were stimulated with TNFα (25 ng/mL) and IFNγ (25 ng/ml) in the presence/absence of increasing concentrations of JAK inhibitors. Data are presented as IL-6 concentrations for each group.

FIG. 7 illustrates the gene expression (MFI) of JAK1, JAK3, and TYK2 in the skin of healthy controls and subjects with hidradenitis suppurativa. Data are presented as JAK1, JAK3, or TYK2 gene expression levels for each Healthy Control (n=4) and Hidradenitis Suppurativa (n=41) subject.

FIG. 8 illustrates the gene expression (MFI) of STAT1, STAT2, and STAT3 in the skin of healthy controls and subjects with hidradenitis suppurativa. Data are presented as STAT1, STAT2, or STAT3 gene expression levels for each Healthy Control (n=4) and Hidradenitis Suppurativa (n=41) subject.

FIG. 9 illustrates the gene expression (MFI) of IRAK1, IRAK2, and IRAK4 in the skin of healthy controls and subjects with hidradenitis suppurativa. Data are presented as IRAK1, IRAK2, or IRAK4 gene expression levels for each Healthy Control (n=4) and Hidradenitis Suppurativa (n=41) subject.

### DETAILED DESCRIPTION

The present application provides, inter alia, a method of treating hidradenitis suppurativa in a patient in need thereof, comprising administering a therapeutically effective amount of compound which inhibits JAK1 and/or JAK2, or a pharmaceutically acceptable salt thereof.

The method described herein utilize compound or salts that are inhibitors of JAK1 and/or JAK2. In some embodiments, the compound is:

ruxolitinib;

ruxolitinib, wherein one or more hydrogen atoms are replaced by deuterium atoms;

{1-{1-[3-Fluoro-2-(trifluoromethyl)isonicotinoyl]piperidin-4-yl}-3-[4-(7H-pyrrolo[2,3-d]pyrimidin-4-yl)-1H-pyrazol-1-yl]azetidin-3-yl}acetonitrile;

4-{3-(Cyanomethyl)-3-[4-(7H-pyrrolo[2,3-d]pyrimidin-4-yl)-1H-pyrazol-1-yl]azetidin-1-yl}-N-[4-fluoro-2-(trifluoromethyl)phenyl]piperidine-1-carboxamide;

[3-[4-(7H-pyrrolo[2,3-d]pyrimidin-4-yl)-1H-pyrazol-1-yl]-1-(1-{[2-(trifluoromethyl)pyrimidin-4-yl]carbonyl}piperidin-4-yl)azetidin-3-yl]acetonitrile;

4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide;

((2R,5S)-5-{2-[(1R)-1-hydroxyethyl]-1H-imidazo[4,5-d]thieno[3,2-b]pyridin-1-yl}tetrahydro-2H-pyran-2-yl)acetonitrile;

3-[1-(6-chloropyridin-2-yl)pyrrolidin-3-yl]-3-[4-(7H-pyrrolo[2,3-d]pyrimidin-4-yl)-1H-pyrazol-1-yl]propanenitrile;

3-(1-[1,3]oxazolo[5,4-b]pyridin-2-ylpyrrolidin-3-yl)-3-[4-(7H-pyrrolo[2,3-d]pyrimidin-4-yl)-1H-pyrazol-1-yl]propanenitrile;

4-[(4-{3-cyano-2-[4-(7H-pyrrolo[2,3-d]pyrimidin-4-yl)-1H-pyrazol-1-yl]propyl}piperazin-1-yl)carbonyl]-3-fluorobenzonitrile;

4-[(4-{3-cyano-2-[3-(7H-pyrrolo[2,3-d]pyrimidin-4-yl)-1H-pyrrol-1-yl]propyl}piperazin-1-yl)carbonyl]-3-fluorobenzonitrile;

[trans-1-[4-(7H-pyrrolo[2,3-d]pyrimidin-4-y])-1H-pyrazol-1-yl]-3-(4-{[2-(trifluoromethyl)pyrimidin-4-yl]carbonyl}piperazin-1-yl)cyclobutyl}acetonitrile;

{trans-3-(4-{[4-[(3-hydroxyazetidin-1-yl)methyl]-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;

{trans-3-(4-{[4-{[(2S)-2-(hydroxymethyl)pyrrolidin-1-yl]methyl}-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;

{trans-3-(4-{[4-{[(2R)-2-(hydroxymethyl)pyrrolidin-1-yl]methyl}-6-(trifluoromethyl)pyridin-2-yl]oxy}piperidin-1-yl)-1-[4-(7H-pyrrolo[2,3-d]pyrimidin-4-y1)-1H-pyrazol-1-yl]cyclobutyl}acetonitrile;

4-(4-{3-[(dimethylamino)methyl]-5-fluorophenoxy}piperidin-1-yl)-3-[4-(7H-pyrrolo[2,3-d]pyrimidin-4-yl)-1H-pyrazol-1-yl]butanenitrile;

5-{3-(cyanomethyl)-3-[4-(7H-pyrrolo[2,3-d]pyrimidin-4-yl)-1H-pyrazol-1-yl]azetidin-1-yl}-N-isopropylpyrazine-2-carboxamide;

4-{3-(cyanomethyl)-3-[4-(7H-pyrrolo[2,3-d]pyrimidin-4-yl)-1H-pyrazol-1-yl]azetidin-1-yl}-2,5-difluoro-N-[(1S)-2,2,2-trifluoro-1-methylethyl]benzamide;

5-{3-(cyanomethyl)-3-[4-(1H-pyrrolo[2,3-b]pyridin-4-yl)-1H-pyrazol-1-yl]azetidin-1-yl}-N-isopropylpyrazine-2-carboxamide;

{1-(cis-4-{[6-(2-hydroxyethyl)-2-(trifluoromethyl)pyrimidin-4-yl]oxy}cyclohexyl)-3-[4-(7H-pyrrolo[2,3-d]pyrimidin-4-yl)-1H-pyrazol-]-yl]azetidin-3-yl}acetonitrile;

{1-(cis-4-{[4-[(ethylamino)methyl]-6-(trifluoromethyl)pyridin-2-yl]oxy}cyclohexyl)-3-[4-(7H-pyrrolo[2,3-d]pyrimidin-4-yl)-1H-pyrazol-1-yl]azetidin-3-yl}acetonitrile;

{1-(cis-4-{[4-(1-hydroxy-1-methylethyl)-6-(trifluoromethyl)pyridin-2-yl]oxy}cyclohexyl)-3-[4-(7H-pyrrolo[2,3-d]pyrimidin-4-yl)-1H-pyrazol-1-yl]azetidin-3-yl}acetonitrile;

{1-(cis-4-{[4-{[(3R)-3-hydroxypyrrolidin-1-yl]methyl}-6-(trifluoromethyl)pyridin-2-yl]oxy}cyclohexyl)-3-[4-(7H-pyrrolo[2,3-d]pyrimidin-4-yl)-1H-pyrazol-1-yl]azetidin-3-yl}acetonitrile;

{1-(cis-4-{[4-{[(3S)-3-hydroxypyrrolidin-1-yl]methyl}-6-(trifluoromethyl)pyridin-2-yl]oxy}cyclohexyl)-3-[4-(7H-pyrrolo[2,3-d]pyrimidin-4-y])-1H-pyrazol-1-yl]azetidin-3-yl}acetonitrile;

{trans-3-(4-{[4-({[(1S)-2-hydroxy-1-methylethyl]amino}methyl)-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;

{trans-3-(4-{[4-({[(2R)-2-hydroxypropyl]amino}methyl)-6-(trifluoromethyl)pyridin-2-yl]oxy}piperidin-1-yl)-1-[4-(7H-pyrrolo[2,3-d]pyrimidin-4-y1)-1H-pyrazol-1-yl]cyclobutyl}acetonitrile{trans-3-(4-{[4-({[(2S)-2-hydrox;

ypropyl]amino}methyl)-6-(trifluoromethyl)pyridin-2-yl]oxy}piperidin-1-yl)-1-[4-(7H-pyrrolo[2,3-d]pyrimidin-4-yl)-1H-pyrazol-1-yl]cyclobutyl}acetonitrile;

{trans-3-(4-{[4-(2-hydroxyethyl)-6-(trifluoromethyl)pyridin-2-yl]oxy}piperidin-1-yl)-1-[4-(7H-pyrrolo[2,3-d]pyrimidin-4-y])-1H-pyrazol-1-yl]cyclobutyl}acetonitrile;

or a pharmaceutically acceptable salt of any of the aforementioned.

In some embodiments, the compound or salt is selective for JAK1 and JAK2 over JAK3 and TYK2. In some embodiments, the compound is 3-cyclopentyl-3-[4-(7H-pyrrolo[2,3-d]pyrimidin-4-yl)-1H-pyrazol-1-yl]propanenitrile, or a pharmaceutically acceptable salt thereof In some embodiments, the compound is (3R)-3-cyclopentyl-3-[4-(7H-pyrrolo[2,3-d]pyrimidin-4-yl)-1H-pyrazol-1-yl]propanenitrile (ruxolitinib), or a pharmaceutically acceptable salt thereof Ruxolitinib has an IC₅₀ of less than 10 nM at 1 mM ATP (assay A) at JAK1 and JAK2. 3-Cyclopentyl-3-[4-(7H-pyrrolo[2,3-d]pyrimidin-4-yl)-1H-pyrazol-1-yl]propanenitrile and ruxolitinib can be made by the procedure described in U.S. Pat. No. 7,598,257 (Example 67), filed Dec. 12, 2006, which is incorporated herein by reference in its entirety. In some embodiments, the inhibitor of JAK1 and/or JAK2 is (3R)-3-cyclopentyl-3-[4-(7H-pyrrolo[2,3-d]pyrimidin-4-yl)-1H-pyrazol-1-yl]propanenitrile phosphoric acid salt. The phosphoric acid salt can be made as described in U.S. Pat. No. 8,722,693, which is incorporated herein by reference in its entirety.

In some embodiments, the compound or salt is a JAK1 inhibitor. In some embodiments, the compound or salt is selective for JAK1 over JAK2, JAK3 and TYK2. For example, some of the compounds described herein, or a pharmaceutically acceptable salt thereof, preferentially inhibit JAK1 over one or more of JAK2, JAK3, and TYK2. JAK1 plays a central role in a number of cytokine and growth factor signaling pathways that, when dysregulated, can result in or contribute to disease states. For example, IL-6 levels are elevated in rheumatoid arthritis, a disease in which it has been suggested to have detrimental effects (Fonesca, et al., Autoimmunity Reviews, 8:538-42, 2009). Because IL-6 signals, at least in part, through JAK1, IL-6 can be indirectly through JAK1 inhibition, resulting in potential clinical benefit (Guschin, et al. Embo J 14:1421, 1995; Smolen, et al. Lancet 371:987, 2008). Moreover, in some cancers JAK1 is mutated resulting in constitutive undesirable tumor cell growth and survival (Mullighan, Proc Natl Acad Sci U S A. 106:9414-8, 2009; Flex, J Exp Med. 205:751-8, 2008). In other autoimmune diseases and cancers, elevated systemic levels of inflammatory cytokines that activate JAK1 may also contribute to the disease and/or associated symptoms. Therefore, patients with such diseases may benefit from JAK1 inhibition. Selective inhibitors of JAK1 may be efficacious while avoiding unnecessary and potentially undesirable effects of inhibiting other JAK kinases.

Hidradenitis suppurativa is characterized by significant skin inflammation; however, there are limited publications outlining the inflammation (Hoffman et al., PLOS One, Sep. 28, 2018, https://doi.org/10.1371/journal.pone.0203672). Presented herein are Examples that support the hypothesis that the inflammation is driven, in large part, by JAK/STAT mediated pathways. Examples C, D and E illustrate elevated levels of JAK/STAT gene expression in the skin of HS patients compared to healthy skin. Further, Examples C, D and E show that pro-inflammatory cytokines which are known to be elevated in HS (TNF-alpha and IFN-gamma) induce the JAK/STAT pathway in cultured keratinocytes and that this induction can be reduced by the addition of JAK inhibitors. Therefore, patients with HS may benefit from JAK1 inhibition. Selective inhibitors of JAK1 may be efficacious while avoiding unnecessary and potentially undesirable effects of inhibiting other JAK kinases.

In some embodiments, the compound or salt inhibits JAK1 preferentially over JAK2 (e.g., have a JAK2/JAK1 IC₅₀ ratio >1). In some embodiments, the compounds or salts are about 10-fold more selective for JAK1 over JAK2. In some embodiments, the compounds or salts are about 3-fold, about 5-fold, about 10-fold, about 15-fold, or about 20-fold more selective for JAK1 over JAK2 as calculated by measuring IC56 at 1 mM ATP (see Example A).

In some embodiments, the JAK1 inhibitor is a compound of Table 1, or a pharmaceutically acceptable salt thereof. The compounds in Table 1 are selective JAK1 inhibitors (selective over JAK2, JAK3, and TYK2). The IC50 values obtained by the method of Example A at 1 mM ATP are shown in Table 1.

TABLE 1

| | | | | JAK1 | |
|---|---|---|---|---|---|
| Comp. | | | | IC₅₀ | JAK2/ |
| No. | Prep. | Name | Structure | (nM) | JAK1 |
| 1 | US 2011/ 0224190 (Example 1) | {1-{1-[3-Fluoro-2- (trifluoromethyl)isonico- tinoyl]piperidin-4-yl}-3- [4-(7H-pyrrolo[2,3- d]pyrimidin-4-yl)-1H- pyrazol-1-yl]azetidin-3- yl}acetonitrile | 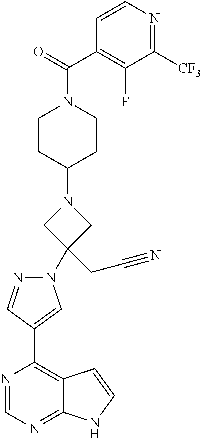 [SMILES: N#CCC1(n2cc(-c3ncnc4[nH]ccc34)cn2)CN(C2CCN(C(=O)c3ccnc(C(F)(F)F)c3F)CC2)C1] | + | >10 |
| 2 | US 2011/ 0224190 (Example 154) | 4-{3-(Cyanomethyl)-3- [4-(7H-pyrrolo[2,3- d]pyrimidin-4-yl)-1H- pyrazol-1-yl]azetidin-1- yl}-N-[4-fluoro-2- (trifluoromethyl)phenyl] piperidine-1- carboxamide | 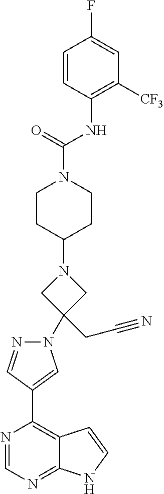 [SMILES: N#CCC1(n2cc(-c3ncnc4[nH]ccc34)cn2)CN(C2CCN(C(=O)Nc3ccc(F)cc3C(F)(F)F)CC2)C1] | + | >10 |
| 3 | US 2011/ 0224190 (Example 85) | [3-[4-(7H-pyrrolo[2,3- d]pyrimidin-4-yl)-1H- pyrazol-1-yl]-1-(1-{[2- (trifluoromethyl) pyrimidin-4- yl]carbonyl}piperidin-4- yl)azetidin-3- yl]acetonitrile | 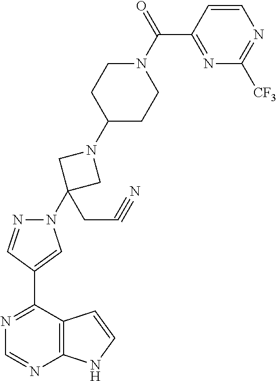 [SMILES: N#CCC1(n2cc(-c3ncnc4[nH]ccc34)cn2)CN(C2CCN(C(=O)c3ccnc(C(F)(F)F)n3)CC2)C1] | + | >10 |
| 4 | US 2014/ 0343030 (Example 7) | 4-[3-(cyanomethyl)-3- (3′,5′-dimethyl-1H,1′H- 4,4′-bipyrazol-1- yl)azetidin-1-yl]-2,5- difluoro-N-[(1S)-2,2,2- trifluoro-1- methylethyl]benzamide | 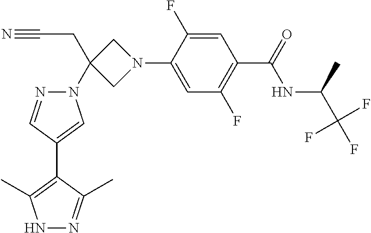 [SMILES: Cc1n[nH]c(C)c1-c1cnn(C2(CC#N)CN(c3cc(F)c(C(=O)N[C@@H](C)C(F)(F)F)cc3F)C2)c1] | +++ | >10 |
| 5 | US 2014/ 0121198 (Example 20) | ((2R,5S)-5-{2-[(1R)-1- hydroxyethyl]-1H- imidazo[4,5- d]thieno[3,2-b]pyridin- 1-yl}tetrahydro-2H- pyran-2-yl)acetonitrile | 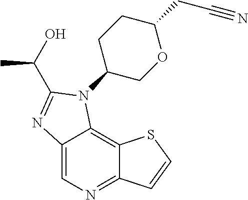 [SMILES: C[C@@H](O)c1nc2cnc3ccsc3c2n1[C@H]1CC[C@H](CC#N)OC1] | ++ | >10 |
| 6 | US 2010/ 0298334 (Example 2)^{a} | 3-[1-(6-chloropyridin-2- yl)pyrrolidin-3-yl]-3-[4- (7H-pyrrolo[2,3- d]pyrimidin-4-yl)-1H- pyrazol-1- yl]propanenitrile | 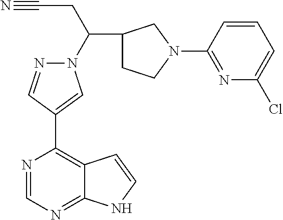 [SMILES: N#CCC(C1CCN(c2cccc(Cl)n2)C1)n1cc(-c2ncnc3[nH]ccc23)cn1] | + | >10 |
| 7 | US 2010/ 0298334 (Example 13c) | 3-(1-[1,3]oxazolo[5,4- b]pyridin-2- ylpyrrolidin-3-yl)-3-[4- (7H-pyrrolo[2,3- d]pyrimidin-4-yl)-1H- pyrazol-1- yl]propanenitrile | 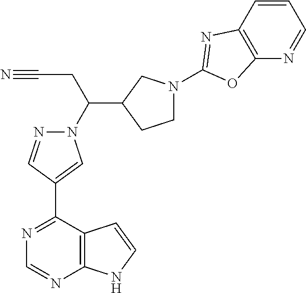 [SMILES: N#CCC(C1CCN(c2nc3cccnc3o2)C1)n1cc(-c2ncnc3[nH]ccc23)cn1] | + | >10 |
| 8 | US 2011/ 0059951 (Example 12) | 4-[(4-{3-cyano-2-[4- (7H-pyrrolo[2,3- d]pyrimidin-4-yl)-1H- pyrazol-1- yl]propyl}piperazin-1- yl)carbonyl]-3- fluorobenzonitrile | 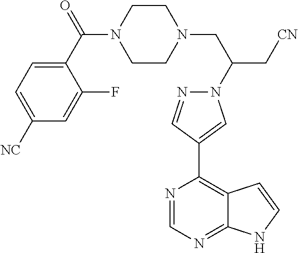 [SMILES: N#CCC(CN1CCN(C(=O)c2ccc(C#N)cc2F)CC1)n1cc(-c2ncnc3[nH]ccc23)cn1] | + | >10 |
| 9 | US 2011/ 0059951 (Example 13) | 4-[(4-{3-cyano-2-[3- (7H-pyrrolo[2,3- d]pyrimidin-4-yl)-1H- pyrrol-1- yl]propyl}piperazin-1- yl)carbonyl]-3- fluorobenzonitrile | 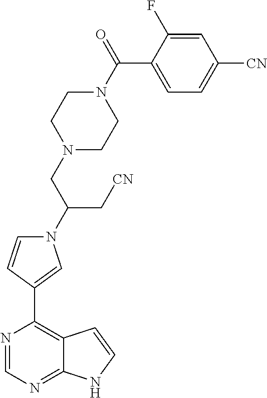 [SMILES: N#CCC(CN1CCN(C(=O)c2ccc(C#N)cc2F)CC1)n1ccc(-c2ncnc3[nH]ccc23)c1] | + | >10 |
| 10 | US 2012/ 0149681 (Example 7b) | [trans-1-[4-(7H- pyrrolo[2,3- d]pyrimidin-4-yl)-1H- pyrazol-1-yl]-3-(4- {[2-(trifluoromethyl) pyrimidin-4- yl]carbonyl} piperazin-1- yl)cyclobutyl] acetonitrile | 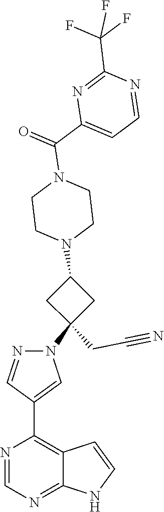 [SMILES: N#CC[C@]1(n2cc(-c3ncnc4[nH]ccc34)cn2)C[C@H](N2CCN(C(=O)c3ccnc(C(F)(F)F)n3)CC2)C1] | + | >10 |
| 11 | US 2012/ 0149681 (Example 157) | {trans-3-(4-{[4-[(3- hydroxyazetidin-1- yl)methyl]-6- (trifluoromethyl)pyridin- 2-yl]oxy}piperidin-1- yl)-1-[4-(7H- pyrrolo[2,3- d]pyrimidin-4-yl)-1H- pyrazol-1- yl]cyclobutyl} acetonitrile | 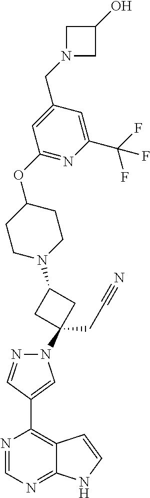 [SMILES: N#CC[C@]1(n2cc(-c3ncnc4[nH]ccc34)cn2)C[C@H](N2CCC(Oc3cc(CN4CC(O)C4)cc(C(F)(F)F)n3)CC2)C1] | + | >10 |
| 12 | US 2012/ 0149681 (Example 161) | {trans-3-(4-{[4-{[(2S)- 2-(hydroxymethyl) pyrrolidin-1-yl] methyl}-6- (trifluoromethyl) pyridin-2-yl]oxy} piperidin-1- yl)-1-[4-(7H- pyrrolo[2,3-d] pyrimidin-4-yl)-1H- pyrazol-1-yl] cyclobutyl}acetonitrile | 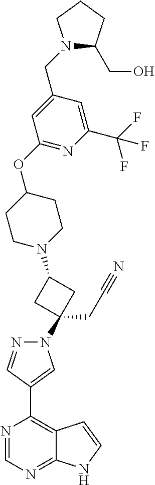 [SMILES: N#CC[C@]1(n2cc(-c3ncnc4[nH]ccc34)cn2)C[C@H](N2CCC(Oc3cc(CN4CCC[C@H]4CO)cc(C(F)(F)F)n3)CC2)C1] | + | >10 |
| 13 | US 2012/ 0149681 (Example 162) | {trans-3-(4-{[4-{[(2R)- 2-(hydroxymethyl) pyrrolidin-1-yl] methyl}-6- (trifluoromethyl) pyridin-2-yl]oxy} piperidin-1- yl)-1-[4-(7H- pyrrolo[2,3-d] pyrimidin-4-yl)-1H- pyrazol-1-yl] cyclobutyl}acetonitrile | 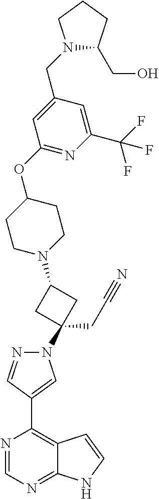 [SMILES: N#CC[C@]1(n2cc(-c3ncnc4[nH]ccc34)cn2)C[C@H](N2CCC(Oc3cc(CN4CCC[C@@H]4CO)cc(C(F)(F)F)n3)CC2)C1] | + | >10 |
| 14 | US 2012/ 0149682 (Example 20)^{b} | 4-(4-{3- [(dimethylamino) methyl]-5- fluorophenoxy} piperidin-1-yl)- 3-[4-(7H- pyrrolo[2,3-d] pyrimidin-4-yl)- 1H-pyrazol-1- yl]butanenitrile | 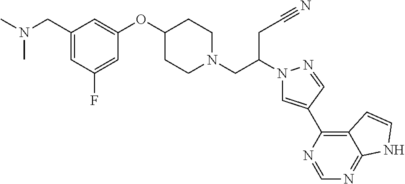 [SMILES: CN(C)Cc1cc(F)cc(OC2CCN(CC(CC#N)n3cc(-c4ncnc5[nH]ccc45)cn3)CC2)c1] | + | >10 |
| 15 | US 2013/ 0018034 (Example 18) | 5-{3-(cyanomethyl)-3- [4-(7H-pyrrolo[2,3- d]pyrimidin-4-yl)-1H- pyrazol-1-yl]azetidin-1- yl}-N- isopropylpyrazine-2- carboxamide | 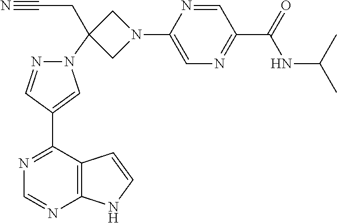 [SMILES: CC(C)NC(=O)c1cnc(N2CC(CC#N)(n3cc(-c4ncnc5[nH]ccc45)cn3)C2)cn1] | + | >10 |
| 16 | US 2013/ 0018034 (Example 28) | 4-{3-(cyanomethyl)-3- [4-(7H-pyrrolo[2,3- d]pyrimidin-4-yl)-1H- pyrazol-1-yl]azetidin-1- yl}-2,5-difluoro-N- [(1S)-2,2,2-trifluoro-1- methylethyl]benzamide | 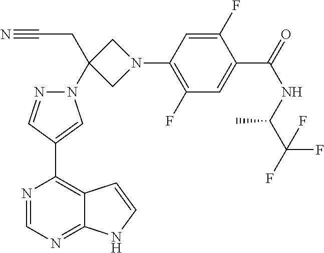 [SMILES: C[C@H](NC(=O)c1cc(F)c(N2CC(CC#N)(n3cc(-c4ncnc5[nH]ccc45)cn3)C2)cc1F)C(F)(F)F] | + | >10 |
| 17 | US 2013/ 0018034 (Example 34) | 5-{3-(cyanomethyl)-3- [4-(1H-pyrrolo[2,3- b]pyridin-4-yl)-1H- pyrazol-1-yl]azetidin- 1-yl}-N- isopropylpyrazine-2- carboxamide | 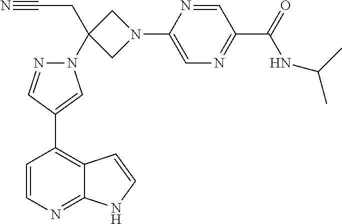 [SMILES: CC(C)NC(=O)c1cnc(N2CC(CC#N)(n3cc(-c4ccnc5[nH]ccc45)cn3)C2)cn1] | + | >10 |
| 18 | US 2013/ 0045963 (Example 45) | {1-(cis-4-{[6-(2- hydroxyethyl)-2- (trifluoromethyl) pyrimidin-4- yl]oxy}cyclohexyl)-3- [4-(7H-pyrrolo[2,3- d]pyrimidin-4-yl)-1H- pyrazol-1-yl] azetidin-3-yl} acetonitrile | 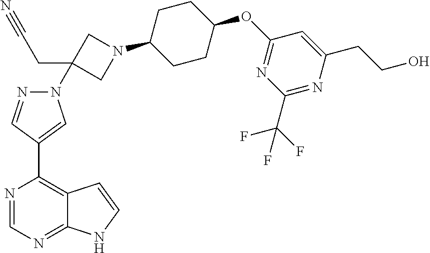 [SMILES: N#CCC1(n2cc(-c3ncnc4[nH]ccc34)cn2)CN([C@H]2CC[C@@H](Oc3cc(CCO)nc(C(F)(F)F)n3)CC2)C1] | + | >10 |
| 19 | US 2013/ 0045963 (Example 65) | {1-(cis-4-{[4- [(ethylamino)methyl]- 6-(trifluoromethyl) pyridin-2-yl]oxy} cyclohexyl)-3-[4- (7H-pyrrolo[2,3-d] pyrimidin-4-yl)-1H- pyrazol-1-yl] azetidin-3-yl} acetonitrile | 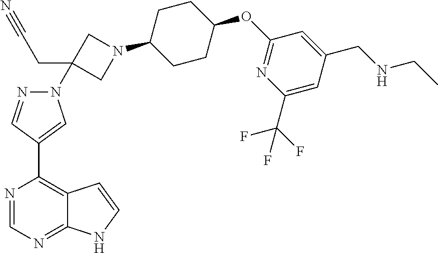 [SMILES: CCNCc1cc(O[C@H]2CC[C@@H](N3CC(CC#N)(n4cc(-c5ncnc6[nH]ccc56)cn4)C3)CC2)nc(C(F)(F)F)c1] | + | >10 |
| 20 | US 2013/ 0045963 (Example 69) | {1-(cis-4-{[4-(1- hydroxy-1- methylethyl)-6- (trifluoromethyl) pyridin-2-yl]oxy} cyclohexyl)-3-[4- (7H-pyrrolo[2,3-d] pyrimidin-4-yl)-1H- pyrazol-1-yl] azetidin-3-yl} acetonitrile | 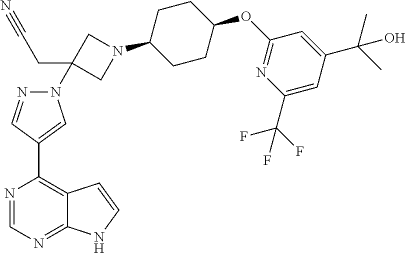 [SMILES: CC(C)(O)c1cc(O[C@H]2CC[C@@H](N3CC(CC#N)(n4cc(-c5ncnc6[nH]ccc56)cn4)C3)CC2)nc(C(F)(F)F)c1] | + | >10 |
| 21 | US 2013/ 0045963 (Example 95) | {1-(cis-4-{[4-{[(3R)-3- hydroxypyrrolidin-1- yl]methyl}-6- (trifluoromethyl) pyridin-2-yl]oxy} cyclohexyl)-3-[4- (7H-pyrrolo[2,3-d] pyrimidin-4-yl)-1H- pyrazol-1-yl] azetidin-3-yl} acetonitrile | 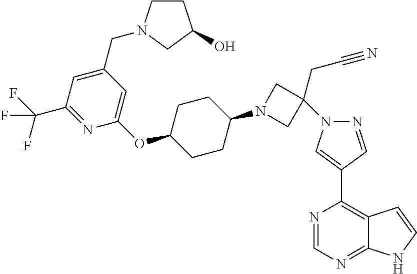 [SMILES: N#CCC1(n2cc(-c3ncnc4[nH]ccc34)cn2)CN([C@H]2CC[C@@H](Oc3cc(CN4CC[C@@H](O)C4)cc(C(F)(F)F)n3)CC2)C1] | + | >10 |
| 22 | US 2013/ 0045963 (Example 95) | {1-(cis-4-{[4-{[(3S)-3- hydroxypyrrolidin-1- yl]methyl}-6- (trifluoromethyl) pyridin-2-yl]oxy} cyclohexyl)-3-[4- (7H-pyrrolo[2,3-d] pyrimidin-4-yl)-1H- pyrazol-1-yl] azetidin-3-yl} acetonitrile | 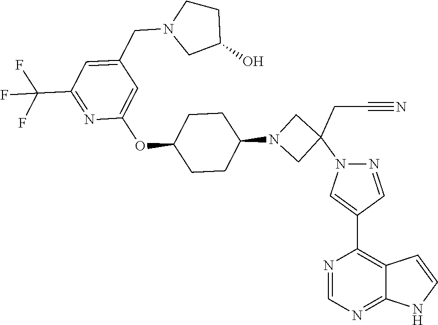 [SMILES: N#CCC1(n2cc(-c3ncnc4[nH]ccc34)cn2)CN([C@H]2CC[C@@H](Oc3cc(CN4CC[C@H](O)C4)cc(C(F)(F)F)n3)CC2)C1] | + | >10 |
| 23 | US 2014/ 0005166 (Example 1) | {trans-3-(4-{[4- ({[(1S)-2-hydroxy-1- methylethyl]amino} methyl)-6- (trifluoromethyl) pyridin-2-yl]oxy} piperidin-1-yl)-1-[4- (7H-pyrrolo[2,3-d] pyrimidin-4-yl)-1H- pyrazol-1-yl] cyclobutyl} acetonitrile | 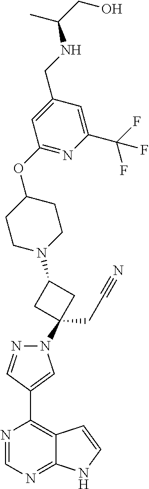 [SMILES: C[C@@H](CO)NCc1cc(OC2CCN([C@H]3C[C@](CC#N)(n4cc(-c5ncnc6[nH]ccc56)cn4)C3)CC2)nc(C(F)(F)F)c1] | + | >10 |
| 24 | US 2014/ 0005166 (Example 14) | {trans-3-(4-{[4- ({[(2R)-2- hydroxypropyl] amino}methyl)-6- (trifluoromethyl) pyridin-2-yl]oxy} piperidin-1-yl)-1-[4- (7H-pyrrolo[2,3-d] pyrimidin-4-yl)-1H- pyrazol-1-yl] cyclobutyl} acetonitrile | 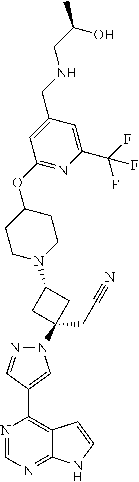 [SMILES: C[C@@H](O)CNCc1cc(OC2CCN([C@H]3C[C@](CC#N)(n4cc(-c5ncnc6[nH]ccc56)cn4)C3)CC2)nc(C(F)(F)F)c1] | + | >10 |
| 25 | US 2014/ 0005166 (Example 15) | {trans-3-(4-{[4- ({[(2S)-2- hydroxypropyl] amino}methyl)-6- (trifluoromethyl) pyridin-2-yl]oxy} piperidin-1-yl)-1-[4- (7H-pyrrolo[2,3-d] pyrimidin-4-yl)-1H- pyrazol-1-yl] cyclobutyl} acetonitrile | 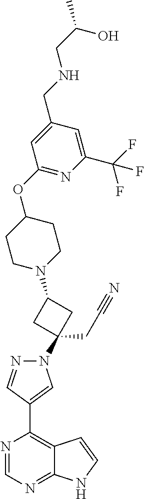 [SMILES: C[C@H](O)CNCc1cc(OC2CCN([C@H]3C[C@](CC#N)(n4cc(-c5ncnc6[nH]ccc56)cn4)C3)CC2)nc(C(F)(F)F)c1] | + | >10 |
| 26 | US 2014/ 0005166 (Example 20) | {trans-3-(4-{[4-(2- hydroxyethyl)-6- (trifluoromethyl) pyridin-2-yl]oxy} piperidin-1-yl)-1-[4- (7H-pyrrolo[2,3-d] pyrimidin-4-yl)-1H- pyrazol-1-yl] cyclobutyl} acetonitrile | 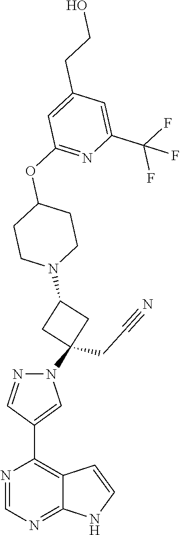 [SMILES: N#CC[C@]1(n2cc(-c3ncnc4[nH]ccc34)cn2)C[C@H](N2CCC(Oc3cc(CCO)cc(C(F)(F)F)n3)CC2)C1] | + | >10 |

+ means <10 nM (see Example A for assay conditions)
++ means ≤100 nM (see Example A for assay conditions)
+++ means ≤300 nM (see Example A for assay conditions)
^{a}Data for enantiomer 1
^{b}Data for enantiomer 2

In some embodiments, the JAK1 inhibitor is {1-{1-[3-fluoro-2-(trifluoromethyl)isonicotinoyl]piperidin-4-yl}-3 [4-(7H-pyrrolo[2,3-d]pyrimidin-4-yl)-1H-pyrazol-1-yl]azetidin-3-yl}acetonitrile, or a pharmaceutically acceptable salt thereof.

In some embodiments, the JAK1 inhibitor is {1-{1-[3-fluoro-2-(trifluoromethyl)isonicotinoyl]piperidin-4-yl}-3[4-(7H-pyrrolo[2,3-d]pyrimidin-4-yl)-1H-pyrazol-1-yl]azetidin-3-yl}acetonitrile adipic acid salt.

The synthesis and preparation of {1-{1-[3-fluoro-2-(trifluoromethyl)isonicotinoyl]piperidin-4-yl}-3[4-(7H-pyrrolo[2,3-d]pyrimidin-4-yl)-1H-pyrazol-1-yl]azetidin-3-yl}acetonitrile and the adipic acid salt of the same can be found, e.g., in US Patent Publ. No. 2011/0224190, filed Mar. 9, 2011, US Patent Publ. No. 2013/0060026, filed Sep. 6, 2012, and US Patent Publ. No. 2014/0256941,filed Mar. 5, 2014, each of which is incorporated herein by reference in its entirety.

In some embodiments, the JAK1 inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide, or a pharmaceutically acceptable salt thereof.

In some embodiments, the JAK1 inhibitor is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide phosphoric acid salt.

In some embodiment, the JAK1 is 4-[3-(cyanomethyl)-3-(3′,5′-dimethyl-1H, 1′H-4,4′-bipyrazol-1-yl)azetidin-1-y]-2,5-difluoro-N-[(1S)-2,2,2-trifluoro-1-methylethyl]benzamide hydrochloric acid salt.

In some embodiment, the JAK1 is 4-[3-(cyanomethyl)-3-(3′,5′-dimethy]-1H, 1′H-4,4′-bipyrazol-1-yl)azetidin-1-yl]-2,5-difluoro-N-[(1S)-2,2,2-trifluoro-1-methylethyl]benzamide hydrobromic acid salt.

In some embodiment, the JAK1 is 4-[3-(cyanomethyl)-3-(3′,S′-dimethyl-1H,1′H-4,4′-bipyrazol-1-yl)azetidin-1-yl]-2,5-difluoro-N-[(1S)-2,2,2-trifluoro-1-methylethyl]benzamide sulfuric acid salt.

The synthesis and preparation of 4-[3-(cyanomethyl)-3-(3′,5′-dimethyl-1H,1′H-4,4′-bipyrazol-1-yl)azetidin-1-y]-2,5-difluoro-N-[(1S)-2,2,2-trifluoro-1-methylethyl]benzamide and the phosphoric acid salt of the same can be found, e.g., in US Patent Publ. No. US 2014/0343030, filed May 16, 2014, which is incorporated herein by reference in its entirety.

In some embodiments, the JAK1 inhibitor is ((2R,5S)-5-{2-[(IR)-1-hydroxyethyl]-1H-imidazo[4,5-d]thieno[3,2-b]pyridin-1-yl}tetrahydro-2H-pyran-2-yl)acetonitrile, or a pharmaceutically acceptable salt thereof.

In some embodiments, the JAK1 inhibitor is ((2R,5S)-5-{2-[(1R)-1-hydroxyethyl]-1H-imidazo[4,5-d]thieno[3,2-b]pyridin-1-yl}tetrahydro-2H-pyran-2-yl)acetonitrile monohydrate.

Synthesis of ((2R,SS)-5-{2-[(1R)-1-hydroxyethyl]-1H-imidazo[4,5-d]thieno [3,2-b]pyridin-1-yl}tetrahydro-2H-pyran-2-yl)acetonitrile and characterization of the anhydrous and monohydrate forms of the same are described in US Patent Publ. No. 2014/0121198, filed Oct. 31, 2013 and US Patent Publ. No. 2015/0344497, filed Apr. 29, 2015, each of which is incorporated herein by reference in its entirety

In some embodiments, the compounds of Table 1 are prepared by the synthetic procedures described in US Patent Publ. No. 2011/0224190, filed Mar. 9, 2011, US Patent Publ. No. 2014/0343030, filed May 16, 2014, US Patent Publ. No. 2014/0121198, filed Oct. 31, 2013, US Patent Publ. No. 2010/0298334, filed May 21, 2010, US Patent Publ. No. 2011/0059951, filed Aug. 31, 2010, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

In some embodiments, JAK1 inhibitor is selected from the compounds, or pharmaceutically acceptable salts thereof, of US Patent Publ. No. 2011/0224190, filed Mar. 9, 2011, US Patent Publ. No. 2014/0343030, filed May 16, 2014, US Patent Publ. No. 2014/0121198, filed Oct. 31, 2013, US Patent Publ. No. 2010/0298334, filed May 21, 2010, US Patent Publ. No. 2011/0059951, filed Aug. 31, 2010, US Patent Publ. No. 2012/0149681, filed Nov. 18, 2011, US Patent Publ. No. 2012/0149682, filed Nov. 18, 2011, US Patent Publ. 2013/0018034, filed Jun. 19, 2012, US Patent Publ. No. 2013/0045963, filed Aug. 17, 2012, and US Patent Publ. No. 2014/0005166, filed May 17, 2013, each of which is incorporated herein by reference in its entirety.

In some embodiments, the JAK1 inhibitor is a compound of Formula I

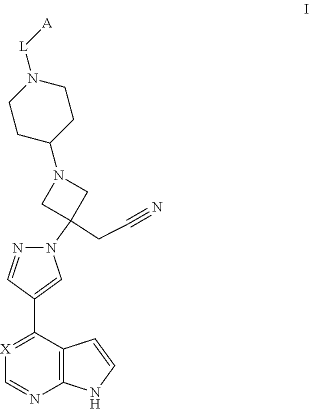

or a pharmaceutically acceptable salt thereof, wherein:

X is N or CH;

L is C(═O) or C(═O)NH;

A is phenyl, pyridinyl, or pyrimidinyl each of which is optionally substituted with 1 or 2 independently selected R¹ groups; and

each R¹ is, independently, fluoro, or trifluoromethyl.

In some embodiments, the compound of Formula I is {1-{1-[3-fluoro-2-(trifluoromethyl)isonicotinoyl]piperidin-4-yl}-3 [4-(7H-pyrrolo[2,3-d]pyrimidin-4-yl)-1H-pyrazol-1-yl]azetidin-3-yl}acetonitrile, or a pharmaceutically acceptable salt thereof.

In some embodiments, the compound of Formula I is 4-{3-(Cyanomethyl)-3-[4-(7H-pyrrolo[2,3-d]pyrimidin-4-yl)-1H-pyrazol-1-yl]azetidin-1-yl}-N-[4-fluoro-2-(trifluoromethyl)phenyl]piperidine-1-carboxamide, or a pharmaceutically acceptable salt thereof.

In some embodiments, the compound of Formula I is [3-[4-(7H-pyrrolo[2,3-d]pyrimidin-4-yl)-1H-pyrazol-1-yl]-1-(1-{[2-(trifluoromethyl)pyrimidin-4-yl]carbonyl}piperidin-4-yl)azetidin-3-yl]acetonitrile, or a pharmaceutically acceptable salt thereof.

In some embodiments, the JAK1 inhibitor is a compound of Formula II

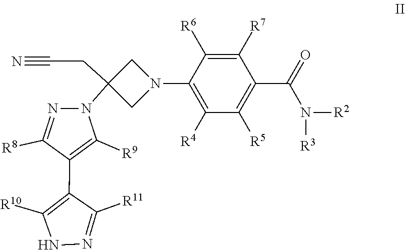

or a pharmaceutically acceptable salt thereof, wherein:

R² is C₁₋₆ alkyl, C₁₋₆ haloalkyl, C₃₋₆ cycloalkyl, or C₃₋₆ cycloalkyl-C₁₋₃ alkyl, wherein said C₁₋₆ alkyl, C₃₋₆ cycloalkyl, and C₃₋₆ cycloalkyl-C₁₋₃ alkyl, are each optionally substituted with 1, 2, or 3 substituents independently selected from fluoro, —CF₃, and methyl;

R³ is H or methyl;

R⁴ is H, F, or CI;

R⁵ is H or F;

R⁶ is H or F;

R⁷ is H or F;

R⁸ is H or methyl;

R⁹ is H or methyl;

R¹⁰ is H or methyl; and

R¹¹ is H or methyl.

In some embodiments, the compound of Formula II is 4-[3-(cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide, or a pharmaceutically acceptable salt thereof.

In some embodiments, the JAK1 inhibitor is a compound of Formula III

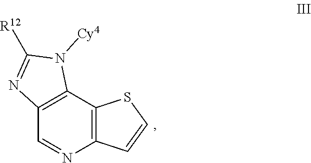

or a pharmaceutically acceptable salt thereof, wherein:

Cy⁴ is a tetrahydro-2H-pyran ring, which is optionally substituted with 1 or 2 groups independently selected from CN, OH, F, Cl, C₁₋₃ alkyl, C₁₋₃ haloalkyl, CN—C₁₋₃ alkyl, HO-C₁₋₃ alkyl, amino, C₁₋₃ alkylamino, and di(C₁₋₃ alkyl)amino, wherein said C₁₋₃ alkyl and di(C₁₋₃ alkyl)amino is optionally substituted with 1, 2, or 3 substituents independently selected from F, Cl, C₁₋₃ alkylaminosulfonyl, and C₁₋₃ alkylsulfonyl; and

R¹² is —CH₂—OH, —CH(CH₃)—OH, or —CH₂—NHSO₂CH₃.

In some embodiments, the compound of Formula III is ((2R,5S)-5-{2-[(1R)-1-hydroxyethyl]-1H-imidazo[4,5-d]thieno[3,2-b]pyridin-1-yl}tetrahydro-2H-pyran-2-yl)acetonitrile, or a pharmaceutically acceptable salt thereof.

In some embodiments, the inhibitor of JAK1 and/or JAK2 is barcitinib, tofacitinib, oclacitinib, filgotinib, gandotinib, lestaurtinib, momelotinib, bacritinib, PF-04965842, upadacitinib, peficitinib, fedratinib, cucurbitacin I, ATI-501 (Aclaris), ATI-502 (Aclaris), JTE052 (Leo Pharma and Japan Tobacco), or CHZ868.

In some embodiments, the inhibitor of JAK1 and/or JAK2 can be an isotopically-labeled compound, or a pharmaceutically acceptable salt thereof. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ²H (also written as D for deuterium), ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms, such as —CD₃ being substituted for —CH₃).

One or more constituent atoms of the compounds described herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et. al. J. Med. Chem. 2011, 54, 201-210; R. Xu et. al. J. Label Compd. Radiopharm. 2015, 58, 308-312). In particular, substitution at one or more metabolism sites may afford one or more of the therapeutic advantages

Accordingly, in some embodiments, the inhibitor of JAK1 and/or JAK2 is a compound, wherein one or more hydrogen atoms in the compound are replaced by deuterium atoms, or a pharmaceutically acceptable salt thereof.

In some embodiments, the inhibitor of JAK1 and/or JAK2 is ruxolitinib, wherein one or more hydrogen atoms are replaced by deuterium atoms, or a pharmaceutically acceptable salt thereof. In some embodiments, the inhibitor of JAK1 and/or JAK2 is any of the compounds in U.S. Pat. No. 9,249,149 (which is incorporated herein by reference in its entirety), or a pharmaceutically acceptable salt thereof. In some embodiments, the inhibitor of JAK1 and/or JAK2 is CTP-543, or a pharmaceutically acceptable salt thereof.

In some embodiments, the compound is a compound of Formula I:

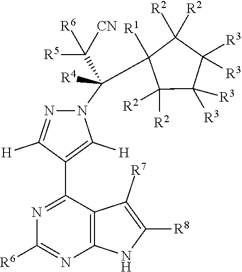

or a pharmaceutically acceptable salt thereof, wherein:

R¹ is selected from H and D;

each R² is independently selected from H and D, provided that each R² attached to a common carbon is the same;

each R³ is independently selected from H and D, provided that each R³ attached to a common carbon is the same,

R⁴ is selected from H and D;

each R⁵ is the same and is selected from H and D; and

R⁶, R⁷, and R⁸ are each independently selected from H and D; provided that when R¹ is H, each R² and each R³ are H, R⁴ is H, and each of R⁶, R⁷, and R⁸ is H, then each R⁵ is D.

In some embodiments, the inhibitor of JAK1 and/or JAK2 is a compound of Formula I selected from the following compounds 100-130 in the table below (wherein R⁶, R⁷, and R⁸ are each H), or a pharmaceutically acceptable salt thereof. In some embodiments, the inhibitor of JAK1 and/or JAK2 is a compound of Formula I selected from the following compounds 200-231 in the table below (wherein R⁶, R⁷, and R⁸ are each D), or a pharmaceutically acceptable salt thereof

| Compound | R¹ | Each R² | Each R³ | R⁴ | Each R⁵ |
|---|---|---|---|---|---|
| 100 | H | H | H | D | H |
| 101 | H | H | H | H | D |
| 102 | H | H | H | D | D |
| 103 | H | H | D | H | H |
| 104 | H | H | D | D | H |
| 105 | H | H | D | H | D |
| 106 | H | H | D | D | D |
| 107 | H | D | H | H | H |
| 108 | H | D | H | D | H |
| 109 | H | D | H | H | D |
| 110 | H | D | H | D | D |
| 111 | H | D | D | H | H |
| 112 | H | D | D | D | H |
| 113 | H | D | D | H | D |
| 114 | H | D | D | D | D |
| 115 | D | H | H | H | H |
| 116 | D | H | H | D | H |
| 117 | D | H | H | H | D |
| 118 | D | H | H | D | D |
| 119 | D | H | D | H | H |
| 120 | D | H | D | D | H |
| 121 | D | H | D | H | D |
| 122 | D | H | D | D | D |
| 123 | D | D | H | H | H |
| 124 | D | D | H | D | H |
| 125 | D | D | H | H | D |
| 126 | D | D | H | D | D |
| 127 | D | D | D | H | H |
| 128 | D | D | D | D | H |
| 129 | D | D | D | H | D |
| 130 | D | D | D | D | D |
| 200 | H | H | H | D | H |
| 201 | H | H | H | H | D |
| 202 | H | H | H | D | D |
| 203 | H | H | D | H | H |
| 204 | H | H | D | D | H |
| 205 | H | H | D | H | D |
| 206 | H | H | D | D | D |
| 207 | H | D | H | H | H |
| 208 | H | D | H | D | H |
| 209 | H | D | H | H | D |
| 210 | H | D | H | D | D |
| 211 | H | D | D | H | H |
| 212 | H | D | D | D | H |
| 213 | H | D | D | H | D |
| 214 | H | D | D | D | D |
| 215 | D | H | H | H | H |
| 216 | D | H | H | D | H |
| 217 | D | H | H | H | D |
| 218 | D | H | H | D | D |
| 219 | D | H | D | H | H |
| 220 | D | H | D | D | H |
| 221 | D | H | D | H | D |
| 222 | D | H | D | D | D |
| 223 | D | D | H | H | H |
| 224 | D | D | H | D | H |
| 225 | D | D | H | H | D |
| 226 | D | D | H | D | D |
| 227 | D | D | D | H | H |
| 228 | D | D | D | D | H |
| 229 | D | D | D | H | D |
| 230 | D | D | D | D | D |
| 231 | H | H | H | H | H |

In some embodiments, the inhibitor of JAK1 and/or JAK2 is baricitinib, wherein one or more hydrogen atoms are replaced by deuterium atoms, or a pharmaceutically acceptable salt thereof. In some embodiments, the inhibitor of JAK1 and/or JAK2 is any of the compounds in U.S. Pat. No. 9,540,367 (which is incorporated herein by reference in its entirety), or a pharmaceutically acceptable salt thereof.

As used herein, the phrase “optionally substituted” means unsubstituted or substituted. As used herein, the term “substituted” means that a hydrogen atom is removed and replaced by a substituent. It is to be understood that substitution at a given atom is limited by valency.

As used herein, the term “Cₙ₋ₘ alkyl”, employed alone or in combination with other terms, refers to a saturated hydrocarbon group that may be straight-chain or branched, having n to m carbon atoms. In some embodiments, the alkyl group contains 1 to 6, or 1 to 3 carbon atoms. Examples of alkyl moieties include, but are not limited to, chemical groups such as methyl, ethyl, n-propyl, isopropyl, n-butyl, isobutyl, sec-butyl, tert-butyl, n-pentyl, 2-methyl-1-butyl, 3-pentyl, n-hexyl, 1,2,2-trimethylpropyl, and the like.

As used herein, the term “alkylene”, employed alone or in combination with other terms, refers to a divalent alkyl linking group, which can be branched or straight-chain, where the two substituents may be attached any position of the alkylene linking group. Examples of alkylene groups include, but are not limited to, ethan-1,2-diyl, propan-1,3-diyl, propan-1,2-diyl, and the like.

As used herein, the term “HO-C₁₋₃-alkyl” refers to a group of formula -alkylene-OH, wherein said alkylene group has 1 to 3 carbon atoms.

As used herein, the term “CN-C₁₋₃ alkyl” refers to a C₁₋₃ alkyl substituted by a cyano group.

As used herein, the term “amino” refers to a group of formula —NH₂.

As used herein, the term “di(C₁₋₃-alkyl)amino” refers to a group of formula —N(alkyl)₂, wherein the two alkyl groups each has, independently, 1 to 3 carbon atoms.

As used herein, the term “C₁₋₃ alkylamino” refers to a group of formula —NH(alkyl), wherein the alkyl group has 1 to 3 carbon atoms.

As used herein, the term “di(C₁₋₃ alkyl) aminosulfonyl” refers to a group of formula —S(O)₂N(alkyl)₂, wherein each alkyl group independently has 1 to 3 carbon atoms.

As used herein, the term “C₁₋₃ alkylsulfonyl” refers to a group of formula —S(O)₂-alkyl, wherein the alkyl group has 1 to 3 carbon atoms.

As used herein, “halo” or “halogen”, employed alone or in combination with other terms, includes fluoro, chloro, bromo, and iodo. In some embodiments, the halo group is fluoro or chloro.

As used herein, the term “Cₙ₋ₘ haloalkyl”, employed alone or in combination with other terms, refers to a Cₙ₋ₘ alkyl group having up to {2(n to m)+1} halogen atoms which may either be the same or different. In some embodiments, the halogen atoms are fluoro atoms. In some embodiments, the alkyl group has 1-6 or 1-3 carbon atoms. Example haloalkyl groups include CF₃, C₂F₅, CHF₂, CCl₃, CHCl₂, C₂Cl₅, and the like. In some embodiments, the haloalkyl group is a fluoroalkyl group.

As used herein, the term “C₁₋₃ fluoroalkyl” refers to a C₁₋₃ alkyl group that may be partially or completely substituted by fluoro atoms.

As used herein, the term “C₃₋₆ cycloalkyl”, employed alone or in combination with other terms, refers to a non-aromatic monocyclic hydrocarbon moiety, having 3-6 carbon atoms, which may optionally contain one or more alkenylene groups as part of the ring structure. One or more ring-forming carbon atoms of a cycloalkyl group can be oxidized to form carbonyl linkages. Exemplary C₃₋₆ cycloalkyl groups include cyclopropyl, cyclobutyl, cyclopentyl, cyclohexyl, cyclopentenyl, cyclohexenyl, cyclohexadienyl, and the like. In some embodiments, the cycloalkyl group is cyclopropyl, cyclobutyl, cyclopentyl, or cyclohexyl.

As used herein, the term “C₃₋₆ cycloalkyl-C₁₋₃ alkyl” refers to a group of formula -C₁₋₃ alkylene-C₃₋₆ cycloalkyl.

The compounds described herein can be asymmetric (e.g., having one or more stereocenters). All stereoisomers, such as enantiomers and diastereomers, are intended unless otherwise indicated. Compounds that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds, and the like can also be present in the compounds described herein, and all such stable isomers are contemplated in the present application. Cis and trans geometric isomers of the compounds of the present application are described and may be isolated as a mixture of isomers or as separated isomeric forms. In some embodiments, the compound has the (R)-configuration. In some embodiments, the compound has the (S)-configuration.

Resolution of racemic mixtures of compounds can be carried out by any of numerous methods known in the art. An example method includes fractional recrystallization using a chiral resolving acid which is an optically active, salt-forming organic acid. Suitable resolving agents for fractional recrystallization methods are, for example, optically active acids, such as the D and L forms of tartaric acid, diacetyltartaric acid, dibenzoyltartaric acid, mandelic acid, malic acid, lactic acid or the various optically active camphorsulfonic acids such as β-camphorsulfonic acid Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane, and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds described herein include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution. For example, it will be recognized that the following pyrazole ring may form two tautomers:

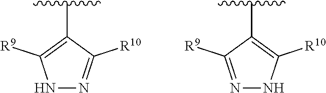

It is intended that the claims cover both tautomers.

All compounds, and pharmaceutically acceptable salts thereof, can be found together with other substances such as water and solvents (e.g. hydrates and solvates) or can be isolated.

In some embodiments, the compounds described herein, or salts thereof, are substantially isolated. By “substantially isolated” is meant that the compound is at least partially or substantially separated from the environment in which it was formed or detected. Partial separation can include, for example, a composition enriched in the compounds described herein. Substantial separation can include compositions containing at least about 50%, at least about 60%, at least about 70%, at least about 80%, at least about 90%, at least about 95%, at least about 97%, or at least about 99% by weight of the compounds described herein, or salt thereof. Methods for isolating compounds and their salts are routine in the art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

The present application also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (ACN) are preferred. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “contacting” refers to the bringing together of indicated moieties in an in vitro system or an in vivo system. For example, “contacting” a JAK with a compound of the invention includes the administration of a compound of the present application to an individual or patient, such as a human, having a JAK, as well as, for example, introducing a compound of the invention into a sample containing a cellular or purified preparation containing the JAK.

As used herein, the term “subject”, “individual” or “patient,” used interchangeably, refers to any animal, including mammals, preferably mice, rats, other rodents, rabbits, dogs, cats, swine, cattle, sheep, horses, or primates, and most preferably humans. In some embodiments, the “subject,” “individual,” or “patient” is in need of said treatment.

In some embodiments, the inhibitors are administered in a therapeutically effective amount. As used herein, the phrase “therapeutically effective amount” refers to the amount of active compound or pharmaceutical agent that elicits the biological or medicinal response that is being sought in a tissue, system, animal, individual or human by a researcher, veterinarian, medical doctor or other clinician.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease; or (3) preventing the disease, condition or disorder in an individual who may be predisposed to the disease, condition or disorder but does not yet experience or display the pathology or symptomatology of the disease. In some embodiments, treating refers to inhibiting or ameliorating the disease. In some embodiments, treating is preventing the disease.

### Combination Therapies

The methods described herein can further comprise administering one or more additional therapeutic agents. The one or more additional therapeutic agents can be administered to a patient simultaneously or sequentially.

In some embodiments, the additional therapeutic agent is an antibiotic. In some embodiments, the antibiotic is clindamycin, doxycycline, minocycline, trimethoprim-sulfamethoxazole, erythromycin, metronidazole, rifampin, moxifloxacin, dapsone, or a combination thereof. In some embodiments, the antibiotic is clindamycin, doxycycline, minocycline, trimethoprim-sulfamethoxazole, or erythromycin in combination with metronidazole. In some embodiments, the antibiotic is a combination of rifampin, moxifloxacin, and metronidazole. In some embodiments, the antibiotic is a combination of moxifloxacin and rifampin.

In some embodiments, the additional therapeutic agent is a retinoid. In some embodiments, the retinoid is etretinate, acitretin, or isotretinoin.

In some embodiments, the additional therapeutic agent is a steroid. In some embodiments, the additional therapeutic agent is a corticosteroid. In some embodiments, the steroid is such as triamcinolone, dexamethasone, fluocinolone, cortisone, prednisone, prednisolone, or flumetholone.

In some embodiments, the additional therapeutic agent is an anti-TNF-alpha agent. In some embodiments, the anti-TNF-alpha agent is an anti-TNF-alpha antibody. In some embodiments, the anti-TNF-alpha agent is infliximab or etanercept, or adalimumab.

In some embodiments, the additional therapeutic agent is an immunosuppressant. In some embodiments, the immunosuppressant is methotrexate or cyclosporin A. In some embodiments, the immunosuppressant is mycophenolate mofetil or mycophenolate sodium.

In some embodiments, the additional therapeutic agent is finasteride, metformin, adapalene or azelaic acid.

In some embodiments, the method further comprises administering an additional therapeutic agent selected from IMiDs, an anti-IL-6 agent, a hypomethylating agent, and a biologic response modifier (BRM).

Generally, a BRM is a substances made from living organisms to treat disease, which may occur naturally in the body or may be made in the laboratory. Examples of BRMs include IL-2, interferon, various types of colony-stimulating factors (CSF, GM-CSF, G-CSF), monoclonal antibodies such as abciximab, etanercept, infliximab, rituximab, trasturzumab, and high dose ascorbate.

In some embodiments, the hypomethylating agent is a DNA methyltransferase inhibitor. In some embodiments, the DNA methyltransferase inhibitor is selected from 5 azacytidine and decitabine.

Generally, IMiDs are as immunomodulatory agents. In some embodiments, the IMiD is selected from thalidomide, lenalidomide, pomalidomide, CC-11006, and CC-10015.

In some embodiments, the method further comprises administering an additional therapeutic agent selected from anti-thymocyte globulin, recombinant human granulocyte colony-stimulating factor (G CSF), granulocyte-monocyte CSF (GM-CSF), an erythropoiesis-stimulating agent (ESA), and cyclosporine.

In some embodiments, the method further comprises administering an additional JAK inhibitor to the patient. In some embodiments, the additional JAK inhibitor is barcitinib, tofacitinib, oclacitinib, filgotinib, gandotinib, lestaurtinib, momelotinib, bacritinib, PF-04965842, upadacitinib, peficitinib, fedratinib, cucurbitacin I, or CHZ868.

One or more additional pharmaceutical agents such as, for example, anti-inflammatory agents, immunosuppressants, as well as PI3Kδ, mTor, Bcr-Abl, Flt-3, RAF and FAK kinase inhibitors such as, for example, those described in WO 2006/056399, which is incorporated herein by reference in its entirety, or other agents can be used in combination with the compounds described herein for treatment of JAK-associated diseases, disorders or conditions. The one or more additional pharmaceutical agents can be administered to a patient simultaneously or sequentially.

Example Bcr-Abl inhibitors include the compounds, and pharmaceutically acceptable salts thereof, of the genera and species disclosed in U.S. Pat. No. 5,521,184, WO 04/005281, and U.S. Ser. No. 60/578,491, all of which are incorporated herein by reference in their entirety.

Example suitable Flt-3 inhibitors include compounds, and their pharmaceutically acceptable salts, as disclosed in WO 03/037347, WO 03/099771, and WO 04/046120, all of which are incorporated herein by reference in their entirety.

Example suitable RAF inhibitors include compounds, and their pharmaceutically acceptable salts, as disclosed in WO 00/09495 and WO 05/028444, both of which are incorporated herein by reference in their entirety.

Example suitable FAK inhibitors include compounds, and their pharmaceutically acceptable salts, as disclosed in WO 04/080980, WO 04/056786, WO 03/024967, WO 01/064655, WO 00/053595, and WO 01/014402, all of which are incorporated herein by reference in their entirety.

In some embodiments, one or more of the compounds of the invention can be used in combination with one or more other kinase inhibitors including imatinib, particularly for treating patients resistant to imatinib or other kinase inhibitors.

In some embodiments, the additional therapeutic agent is fluocinolone acetonide (Retisert®), or rimexolone (AL-2178, Vexol, Alcon).

In some embodiments, the additional therapeutic agent is cyclosporine (Restasis®).

In some embodiments, the additional therapeutic agent is selected from Dehydrex™ (Holles Labs), Civamide (Opko), sodium hyaluronate (Vismed, Lantibio/TRB Chemedia), cyclosporine (ST-603, Sirion Therapeutics), ARG101(T) (testosterone, Argentis), AGR1012(P) (Argentis), ecabet sodium (Senju-Ista), gefarnate (Santen), 15-(s)-hydroxyeicosatetraenoic acid (15(S)-HETE), cevilemine, doxycycline (ALTY-0501, Alacrity), minocycline, iDestrin™ (NP50301, Nascent Pharmaceuticals), cyclosporine A (Nova22007, Novagali), oxytetracycline (Duramycin, MOLI1901, Lantibio), CF101 (2S,3S,4R,5R)-3,4-dihydroxy-5-[6-[(3-iodophenyl)methylamino]purin-9-yl]-N-methyl-oxolane-2-carbamyl, Can-Fite Biopharma), voclosporin (LX212 or LX214, Lux Biosciences), ARG103 (Agentis), RX-10045 (synthetic resolvin analog, Resolvyx), DYN15 (Dyanmis Therapeutics), rivoglitazone (DE011, Daiichi Sanko), TB4 (RegeneRx), OPH-01 (Ophtalmis Monaco), PCS101 (Pericor Science), REV1-31 (Evolutec), Lacritin (Senju), rebamipide (Otsuka-Novartis), OT-551 (Othera), PAI-2 (University of Pennsylvania and Temple University), pilocarpine, tacrolimus, pimecrolimus (AMS981, Novartis), loteprednol ctabonate, rituximab, diquafosol tetrasodium (INS365, Inspire), KLS-0611 (Kissei Pharmaceuticals), dehydroepiandrosterone, anakinra, efalizumab, mycophenolate sodium, etanercept (Embrel®), hydroxychloroquine, NGX267 (TorreyPines Therapeutics), actemra, gemcitabine, oxaliplatin, L-asparaginase, or thalidomide.

In some embodiments, the additional therapeutic agent is an anti-angiogenic agent, cholinergic agonist, TRP-1 receptor modulator, a calcium channel blocker, a mucin secretagogue, MUCI stimulant, a calcineurin inhibitor, a corticosteroid, a P2Y2 receptor agonist, a muscarinic receptor agonist, an mTOR inhibitor, another JAK inhibitor, Bcr-Abl kinase inhibitor, Flt-3 kinase inhibitor, RAF kinase inhibitor, and FAK kinase inhibitor such as, for example, those described in WO 2006/056399, which is incorporated herein by reference in its entirety. In some embodiments, the additional therapeutic agent is a tetracycline derivative (e.g., minocycline or doxycline). In some embodiments, the additional therapeutic agent binds to FKBP12.

In some embodiments, the additional therapeutic agent is an alkylating agent or DNA cross-linking agent; an anti-metabolite/demethylating agent (e.g., 5-flurouracil, capecitabine or azacitidine); an anti-hormone therapy (e.g., hormone receptor antagonists, SERMs, or aromotase inhibitor); a mitotic inhibitor (e.g. vincristine or paclitaxel); an topoisomerase (I or II) inhibitor (e.g. mitoxantrone and irinotecan); an apoptotic inducers (e.g. ABT-737); a nucleic acid therapy (e.g. antisense or RNAi); nuclear receptor ligands (e.g., agonists and/or antagonists: all-trans retinoic acid or bexarotene); epigenetic targeting agents such as histone deacetylase inhibitors (e.g. vorinostat), hypomethylating agents (e.g. decitabine); regulators of protein stability such as Hsp90 inhibitors, ubiquitin and/or ubiquitin like conjugating or deconjugating molecules; or an EGFR inhibitor (erlotinib).

In some embodiments, the additional therapeutic agent includes an antibiotic, antiviral, antifungal, anesthetic, anti-inflammatory agents including steroidal and non-steroidal anti-inflammatoirend anti-allergic agents. Examples of suitable medicaments include aminoglycosides such as amikacin, gentamycin, tobramycin, streptomycin, netilmycin, and kanamycin; fluoroquinolones such as ciprofloxacin, norfloxacin, ofloxacin, trovafloxacin, lomefloxacin, levofloxacin, and enoxacin; naphthyridine; sulfonamides; polymyxin; chloramphenicol; neomycin; paramomycin; colistimethate; bacitracin; vancomycin; tetracyclines; rifampin and its derivatives (“rifampins”); cycloserine; beta-lactams; cephalosporins; amphotericins; fluconazole; flucytosine; natamycin; miconazole; ketoconazole; corticosteroids; diclofenac; flurbiprofen; ketorolac; suprofen; cromolyn; lodoxamide; levocabastin; naphazoline; antazoline; pheniramine; or azalide antibiotic.

### Pharmaceutical Formulations and Dosage Forms

When employed as pharmaceuticals, the compounds of the invention can be administered in the form of pharmaceutical compositions. These compositions can be prepared in a manner well known in the pharmaceutical art, and can be administered by a variety of routes, depending upon whether local or systemic treatment is desired and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

In some embodiments, the administration is topical. In some embodiments, the administration is topical administration to the skin.

In some embodiments, the administration is oral.

This invention also includes pharmaceutical compositions which contain, as the active ingredient, the compound of the invention or a pharmaceutically acceptable salt thereof, in combination with one or more pharmaceutically acceptable carriers (excipients). In some embodiments, the composition is suitable for topical administration. In making the compositions of the invention, the active ingredient is typically mixed with an excipient, diluted by an excipient or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, suppositories, sterile injectable solutions, and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g. about 40 mesh.

The compounds of the invention may be milled using known milling procedures such as wet milling to obtain a particle size appropriate for tablet formation and for other formulation types. Finely divided (nanoparticulate) preparations of the compounds of the invention can be prepared by processes known in the art, e.g., see International App. No. WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The formulations can additionally include: lubricating agents such as talc, magnesium stearate, and mineral oil; wetting agents; emulsifying and suspending agents; preserving agents such as methyl- and propylhydroxy-benzoates; sweetening agents; and flavoring agents. The compositions of the invention can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier. In some embodiments, the composition comprises at least one compound described herein, or a pharmaceutically acceptable salt thereof, and at least one component selected from microcrystalline cellulose, lactose monohydrate, hydroxypropyl methylcellulose, and polyethylene oxide. In some embodiments, the composition comprises at least one compound described herein, or a pharmaceutically acceptable salt thereof, and microcrystalline cellulose, lactose monohydrate, and hydroxypropyl methylcellulose. In some embodiments, the composition comprises at least one compound described herein, or a pharmaceutically acceptable salt thereof, and microcrystalline cellulose, lactose monohydrate, and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel K00LV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from about 1 to about 1,000 mg, from about 1 mg to about 100 mg, from 1 mg to about 50 mg, and from about 1 mg to 10 mg of active ingredient. Preferably, the dosage is from about 1 mg to about 50 mg or about 1 mg to about 10 mg of active ingredient. In some embodiments, each dosage contains about 10 mg of the active ingredient. In some embodiments, each dosage contains about 50 mg of the active ingredient. In some embodiments, each dosage contains about 25 mg of the active ingredient. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

In some embodiments, the compositions comprise from about 1 to about 1,000 mg, from about 1 mg to about 100 mg, from 1 mg to about 50 mg, and from about 1 mg to 10 mg of active ingredient. Preferably, the compositions comprise from about 1 mg to about 50 mg or about 1 mg to about 10 mg of active ingredient. One having ordinary skill in the art will appreciate that this embodies compounds or compositions containing about 1 mg to about 10 mg, about 1 mg to about 20 mg, about 1 mg to about 25 mg, about 1 mg to about 50 mg of the active ingredient.

In some embodiments, the dosage of the compound, or a pharmaceutically acceptable salt thereof, is 15, 30, 60 or 90 mg on a free base basis. In some embodiments, the dosage is 15, 30, 60 or 90 mg on a free base basis, of Compound 4, or a pharmaceutically acceptable salt thereof. In some embodiments, the dosage of the compound, or a pharmaceutically acceptable salt thereof, is 15 mg on a free base basis. In some embodiments, the dosage of the compound, or a pharmaceutically acceptable salt thereof, is 30 mg on a free base basis. In some embodiments, the dosage of the compound, or a pharmaceutically acceptable salt thereof, is 60 mg on a free base basis. In some embodiments, the dosage of the compound, or a pharmaceutically acceptable salt thereof, is 90 mg on a free base basis.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present application. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, for example, about 0.1 to about 1000 mg of the active ingredient of the present application.

The tablets or pills of the present application can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

The liquid forms in which the compounds and compositions of the present application can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions in can be nebulized by use of inert gases. Nebulized solutions may be breathed directly from the nebulizing device or the nebulizing device can be attached to a face masks tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, for example, liquid paraffin, polyoxyethylene alkyl ether, propylene glycol, white Vaseline, and the like. Carrier compositions of creams can be based on water in combination with glycerol and one or more other components, e.g. glycerinemonostearate, PEG-glycerinemonostearate and cetylstearyl alcohol. Gels can be formulated using isopropyl alcohol and water, suitably in combination with other components such as, for example, glycerol, hydroxyethyl cellulose, and the like. In some embodiments, topical formulations contain at least about 0.1, at least about 0.25, at least about 0.5, at least about 1, at least about 2, or at least about 5 wt % of the compound of the invention. The topical formulations can be suitably packaged in tubes of, for example, 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration, and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient, and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers, or stabilizers will result in the formation of pharmaceutical salts.

The therapeutic dosage of a compound of the present application can vary according to, for example, the particular use for which the treatment is made, the manner of administration of the compound, the health and condition of the patient, and the judgment of the prescribing physician. The proportion or concentration of a compound of the invention in a pharmaceutical composition can vary depending upon a number of factors including dosage, chemical characteristics (e.g., hydrophobicity), and the route of administration. For example, the compounds of the invention can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

The compositions of the invention can further include one or more additional pharmaceutical agents, examples of which are listed hereinabove.

### Kits

The present application also includes pharmaceutical kits useful, for example, in the treatment and/or prevention of cytokine-related diseases or disorders, such as CRS, which include one or more containers containing a pharmaceutical composition comprising a therapeutically effective amount of a compound described herein. Such kits can further include, if desired, one or more of various conventional pharmaceutical kit components, such as, for example, containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example A: In vitro JAK Kinase Assay

JAK1 inhibitors that can be used for the treatment of cytokine-related diseases or disorders are tested for inhibitory activity of JAK targets according to the following in vitro assay described in Park et al., Analytical Biochemistry 1999, 269, 94-104. The catalytic domains of human JAK1 (a.a. 837-1142), JAK2 (a.a. 828-1132) and JAK3 (a.a. 781-1124) with an N-terminal His tag are expressed using baculovirus in insect cells and purified. The catalytic activity of JAK1, JAK2 or JAK3 was assayed by measuring the phosphorylation of a biotinylated peptide. The phosphorylated peptide was detected by homogenous time resolved fluorescence (HTRF). IC₅₀s of compounds are measured for each kinase in the 40 microL reactions that contain the enzyme, ATP and 500 nM peptide in 50 mM Tris (pH 7.8) buffer with 100 mM NaCl, 5 mM DTT, and 0.1 mg/mL (0.01%) BSA. For the 1 mM IC₅₀ measurements, ATP concentration in the reactions is 1 mM. Reactions are carried out at room temperature for 1 hour and then stopped with 20 μL 45 mM EDTA, 300 nM SA-APC, 6 nM Eu-Py20 in assay buffer (Perkin Elmer, Boston, MA). Binding to the Europium labeled antibody takes place for 40 minutes and HTRF signal was measured on a Fusion plate reader (Perkin Elmer, Boston, MA). The compounds in Table 1 were tested in this assay and shown to have the IC₅₀ values in Table 1

### Example B: Safety and Efficacy Study of JAK1 and/or JAK2 inhibitors in Subjects with Moderate to Severe Hidradenitis Suppurativa

A randomized, double-blind, placebo-control, multicenter study is conducted on men and women aged 18-75 years with moderate (Hurley Stage II) to severe (Hurley Stage III) hidradenitis suppurativa for at least 6 months. Hurley stage I is associated with abscess formation (single or multiple) without sinus tracts and cicatrization. Hurley stage II is associated with recurrent abscesses with tract formation and cicatrization; single or multiple, widely separated lesions. Hurley stage III is associated with diffuse or near-diffuse involvement or multiple interconnected tracts and abscesses across the entire area. Study participants are randomized into 5 groups (about 50 participants per group) and treated with either 15, 30, 60 or 90 mg of an inhibitor of JAK1 and/or JAK2 (e.g., ruxolitinib, Compound 4, or Compound 5, or a pharmaceutically acceptable salt thereof), or placebo. At week 16 (primary endpoint), participants in the placebo group are re-randomized equally to active treatment arms for 8 weeks. The blind is maintained. The primary endpoint is the proportion of subjects achieving Hidradenitis Suppurativa Clinical Response (HiSCR) at week 16.

Secondary endpoints include (1) Proportion of subjects with HiSCR over baseline at each visit; (2) Proportion of subjects achieving abscess and inflammatory nodule (AN) count of 0 to 2 at each visit; (3) Mean change from baseline in HS Pain Numeric Rating Scale1) at each visit; (4) Change in modified Sartorius scale at week 16 and week 24; (5) Change in number of draining fistulas count at each visit; (6) Proportion of subjects requiring lesional rescue treatment through week 24; (7) Number of episodes of lesional rescue treatments through week 24; (8) Population PK of the inhibitor of JAK1 and/or JAK2 (e.g., apparent clearance, apparent volume of distribution); (9) Safety and tolerability assessed by monitoring the frequency, duration, and severity of AEs, physical examination, vital signs, and laboratory data for hematology, serum chemistry, and urinalysis; (10) Change in Dermatology Quality of Life Index (DLQI) assessment; (11) Change in the severity of the disease from baseline as assessed by the IHS43 scoring at each visit; (12) Change in hidradenitis suppurativa quality of life (HiSQOL) assessment at each visit over baseline; and (13) Assessment of the dose/exposure-response on percentage change from baseline in terms of efficacy and safety endpoints during the treatment periods.

HiSCR is defined as at least a 50% reduction in abscess and inflammatory nodule (AN) count with no increase in abscess count and no increase in draining fistula count at week 16 relative to baseline). Pain Numeric Rating Scale is used to assess the worst skin and average skin pain due to HS. Ratings for the 2 items range from 0 (no skin pain) to 10 (skin pain as bad as you can imagine). The assessments are recorded on a daily diary by participants before they go to bed and based on a recall period of the “last 24 hours.” The modified Sartorius Scale is used to quantify the severity of HS. Points are awarded for 12 body areas (left and right axilla, left and right sub/inframammary areas, intermammary area, left and right buttocks, left and right inguino-crural folds, perianal area, perineal area, and other): points awarded for nodule (2 points for each); abscesses (4 points); fistulas (4 points); scar (1 point); and longest distance between two lesions (2-6 points, 0 if no lesions); and if lesions are separated buy normal skin (yes—0 point; no—6 points). The total Sartorius Scale is the sum of the 12 regional scores. Lesional rescue treatment: In the event that an acutely painful lesion requires an immediate intervention, physicians have the option to perform rescue interventions. Only two types of interventions are allowed: (1) injection with intralesional triamcinolone acetonide suspension (up to 30 mg in total at the same visit) and/or (2) incision and drainage. An intervention can occur on maximally two different lesions at the same visit or on the same lesion at two different study visits. The same lesion cannot be treated two times at the same visit. If a subject requires more than two interventions before week 16, then they are discontinued from the study. International Hidradenitis Suppurativa Severity Score System (IHS4): IH4 (points)=(number of nodules×1)+(number of abscesses×2)+(number of draining tunnels [fistulac/sinuses]×4). Mild HS: ≤3 points; Moderate HS: 4-10 points; Severe HS: ≥11 points.

Study treatment 1 (Active) includes an oral tablet containing 15 mg of 4-[3-(Cyanomethyl)-3-(3′,5′-dimethyl-1H,1′H-4,4′-bipyrazol-1-yl)azetidin-1-yl]-2,5-difluoro-N-[(1S)-2,2,2-trifluoro-1-methylethyl]benzamide. Dosing levels include 15 mg (1 tablet), 30 mg (2 tablets), 60 mg (4 tablets) and 90 mg (6 tablets). Study treatment 2 (Placebo) includes an oral tablet placebo.

Blood samples for measurement of plasma concentrations of the inhibitor of JAK1 and/or JAK2 are taken, at least, on weeks 2, 12, 16, 20 and 24 before and after administration of study drug at predose, 1 hour postdose and 2-5 hours post dose time points. At the premature discontinuation visit if the subjects discontinues prior to week 8, a trough PK sample is collected if feasible. The date/time of the last prior dose administration is also be recorded.

Superiority tests of the inhibitor of JAK1 and/or JAK2 at 90, 60, 30 and 15 mg compared with placebo is carried out using the Hochberg procedure at an overall 2-sided α=0.05 level. Comparisons between each active group and placebo at week 16 is performed with a logistic regression. At all dose levels the superiority tests are significant (for example, a 10%, 20%, 30%, 40%, or 50% improvement in HISCR (Hidradenitis Suppurativa Clinical Response)) and demonstrate the efficacy of the inhibitor of JAK1 and/or JAK2 to treat HS. The tests show a reduction in nodules and non-inferiority/superiority compared to placebo.

All secondary and exploratory efficacy measures are evaluated using descriptive statistics. The clinical safety data (vital signs, routine laboratory tests, and AEs) are analyzed using descriptive statistics. Exposure-response (E-R) relationship(s) between plasma JAK1 and/or JAK2 inhibitor PK exposures and efficacy/safety data are determined. An interim analysis to estimate treatment response and facilitate planning for future studies is conducted when at least half of the randomized subjects reach week 16.

### Example C. Interferon-gamma and Tumor Necrosis-alpha Induced Janus Kinase Expression in Keratinocyte and Subsequent Production of Inflammatory Mediators

Transformed human keratinocyte (HaCaT) cells were purchased from AddexBio (Catalog #T0020001) and cultured in Optimized Dulbecco's Modified Eagle's Medium (AddexBio, Catalog #C0003-02) supplemented with 10% Fetal Bovine Serum (Hyclone, Catalog #16140-071) and 1x Penicillin/Streptomycin (Gibco, Catalog #15140-122). When cells reached 80-90% confluency they were washed with 1x DPBS then detached from tissue culture flasks by incubation with 0.25% Trypsin (Gibco, Catalog #25200-056) for 3-5 minutes at 37° C./5% CO₂. Cell culture media was added to trypsinized cells then cell suspension was transferred to a sterile 15 mL centrifuge tube to be spun down for 10 minutes at 1300 rpms. Media containing trypsin was aspirated from the cell pellet and then the pellet was re-suspended in 10 mL of cell culture media. Cells were counted using a Countess II automated cell counter then seeded into tissue culture treated 24 well plates at a concentration of 4×10⁴ cells/mL and incubated for 48 hours at 37° C./5% CO₂. After 48 hours media was removed and replaced with 500 uL of either cell culture media or a combinatory stimulation of Recombinant Human Interferon gamma (R&D Systems, Catalog #285-IF-100) and Recombinant Human Tumor Necrosis Factor alpha (R&D Systems, Catalog #210-TA-020). HaCaT cells treated with the combinatory cytokine stimulation were treated at final concentrations of 10 ng/ml, 25 ng/mL, 50 ng/mL, or 100 ng/ml of each cytokine. Treated plates were mixed by gentle agitation for 30 seconds then incubated for 24 hours at 37° C./5% CO₂. At the end of the 24 hour incubation, media was immediately removed from cach plate.

RNA was isolated from HaCaT cells using the QuantiGene Plex Assay reagents and protocols (Affymetrix, Catalog #QGP-232-M18042302). Cells were washed with 1x DPBS then lysed by incubation with provided QuantiGene lysis buffer for 30 minutes at 50-55° C. Cell lysates were incubated for 18-24 hours at 55°° C. with capture beads and probe set designed to specifically hybridize to mRNA from targets of interest. The panel of 32 targets of interest included housekeeping genes used for the normalization of the results. After the 18-24 hour incubation signal was amplified utilizing branched DNA methodologies, according to the manufacturer's procedures (Affymetrix, Catalog #QGP-232-M18042302). After hybridization and wash steps assay plate was read on the Luminex 200 and data were expressed as Net Median Fluorescence Intensity. Data was then normalized to the Net Median Fluorescence Intensity of the housekeeping gene HPRT1 (Table 2).

TABLE 2
Stimulation of Human Keratinocytes with TNFα and IFNγ Induces
the JAK/STAT Pathway and Pro-Inflammatory Cytokines

| Gene | Treatment | MFI^{a} | p-value |
|---|---|---|---|
| JAK1 | Vehicle | 126.7 ± 6.55 | — |
| | 10 ng/ml TNFα/IFNγ | 178.19 ± 3.41 | <.0001 |
| | 25 ng/ml TNFα/IFNγ | 195.02 ± 3.47 | <.0001 |
| | 50 ng/ml TNFα/IFNγ | 198.23 ± 2.52 | <.0001 |
| | 100 ng/ml TNFα/IFNγ | 207.34 ± 3.91 | <.0001 |
| JAK2 | Vehicle | 21.7 ± 0.53 | — |
| | 10 ng/ml TNFα/IFNγ | 154.13 ± 11.65 | <.0001 |
| | 25 ng/ml TNFα/IFNγ | 174.07 ± 12.34 | <.0001 |
| | 50 ng/ml TNFα/IFNγ | 180.71 ± 13.63 | <.0001 |
| | 100 ng/ml TNFα/IFNγ | 187.94 ± 13.12 | <.0001 |
| JAK3 | Vehicle | 0.1 ± 0.02 | — |
| | 10 ng/ml TNFα/IFNγ | 0.16 ± 0.05 | 0.8111 |
| | 25 ng/ml TNFα/IFNγ | 0.18 ± 0.05 | 0.596 |
| | 50 ng/ml TNFα/IFNγ | 0.33 ± 0.06 | 0.0082 |
| | 100 ng/ml TNFα/IFNγ | 0.28 ± 0.06 | 0.0532 |
| TYK2 | Vehicle | 167.84 ± 2.25 | — |
| | 10 ng/ml TNFα/IFNγ | 240.49 ± 4.4 | <.0001 |
| | 25 ng/ml TNFα/IFNγ | 250.15 ± 3.41 | <.0001 |
| | 50 ng/ml TNFα/IFNγ | 257.24 ± 3.55 | <.0001 |
| | 100 ng/ml TNFα/IFNγ | 265.37 ± 3.1 | <.0001 |
| STAT1 | Vehicle | 484.33 ± 4.52 | — |
| | 10 ng/ml TNFα/IFNγ | 3834.09 ± 65.62 | <.0001 |
| | 25 ng/ml TNFα/IFNγ | 3935.51 ± 66.15 | <.0001 |
| | 50 ng/ml TNFα/IFNγ | 3943.03 ± 63.05 | <.0001 |
| | 100 ng/ml TNFα/IFNγ | 4136.09 ± 67.06 | <.0001 |
| STAT3 | Vehicle | 606.76 ± 11.51 | — |
| | 10 ng/ml TNFα/IFNγ | 1561.14 ± 40.35 | <.0001 |
| | 25 ng/ml TNFα/IFNγ | 1652.97 ± 39.53 | <.0001 |
| | 50 ng/ml TNFα/IFNγ | 1666.52 ± 52.15 | <.0001 |
| | 100 ng/ml TNFα/IFNγ | 1742.81 ± 38.26 | <.0001 |
| STAT4 | Vehicle | 2.27 ± 0.12 | — |
| | 10 ng/ml TNFα/IFNγ | 3.78 ± 0.22 | <.0001 |
| | 25 ng/ml TNFα/IFNγ | 3.84 ± 0.23 | <.0001 |
| | 50 ng/ml TNFα/IFNγ | 3.72 ± 0.25 | <.0001 |
| | 100 ng/ml TNFα/IFNγ | 3.61 ± 0.28 | 0.0003 |
| STAT5A | Vehicle | 1.03 ± 0.1 | — |
| | 10 ng/ml TNFα/IFNγ | 26.06 ± 3.1 | <.0001 |
| | 25 ng/ml TNFα/IFNγ | 28.58 ± 3.23 | <.0001 |
| | 50 ng/ml TNFα/IFNγ | 31.01 ± 3.37 | <.0001 |
| | 100 ng/ml TNFα/IFNγ | 29.61 ± 2.91 | <.0001 |
| STAT6 | Vehicle | 626.95 ± 22 | — |
| | 10 ng/ml TNFα/IFNγ | 1010.38 ± 14.28 | <.0001 |
| | 25 ng/ml TNFα/IFNγ | 1044.97 ± 12.71 | <.0001 |
| | 50 ng/ml TNFα/IFNγ | 1039.59 ± 10.5 | <.0001 |
| | 100 ng/ml TNFα/IFNγ | 1059.01 ± 13.45 | <.0001 |
| IL1A | Vehicle | 156.9 ± 1.89 | — |
| | 10 ng/ml TNFα/IFNγ | 1786.44 ± 31.13 | <.0001 |
| | 25 ng/ml TNFα/IFNγ | 2135.03 ± 66.58 | <.0001 |
| | 50 ng/ml TNFα/IFNγ | 2256.89 ± 90.79 | <.0001 |
| | 100 ng/ml TNFα/IFNγ | 2459.6 ± 106.2 | <.0001 |
| IL6 | Vehicle | 5.89 ± 0.19 | — |
| | 10 ng/ml TNFα/IFNγ | 311.31 ± 38 81 | 0.0002 |
| | 25 ng/ml TNFα/IFNγ | 410.93 ± 52.93 | <.0001 |
| | 50 ng/ml TNFα/IFNγ | 464.27 ± 61.46 | <.0001 |
| | 100 ng/ml TNFα/IFNγ | 519.31 ± 68.04 | <.0001 |

^{a}Data are presented as the mean ± standard error (SEM)

Target proteins of interest in the media were detected and quantified using the ProCarta Multiplex Immunoassay reagents and protocols (Invitrogen, Catalog #EPX450-12171-901). Media was incubated with antibody conjugated beads designed to bind to the epitopes of specific target proteins and identify the bound protein through the bead's distinctive spectral pattern. Biotinylated detection antibodies, designed to bind to different epitopes of the same target proteins, and Streptavidin-PE are added to assay plates to quantify the amount of the target protein. Assay plates were read on the Luminex 200 and data were expressed as Net Median Fluorescence Intensity. The Net Median Fluorescence Intensity values for the antigen standard curve, prepared according to the manufacturer's procedures (Invitrogen, Catalog #EPX450-12171-901) were plotted against the expected concentrations for each standard. The concentration of each protein was extrapolated from the antigen standard curve and concentrations were expressed as pg/mL (Table 3).

TABLE 3
Stimulation of Human Keratinocytes with TNFα and IFNγ
Induces the Pro-Inflammatory Cytokine Production

| Protein | Treatment | pg/mL^{a} | p-value |
|---|---|---|---|
| IL-1α | Vehicle | 0.37 ± 0.05 | — |
| | 10 ng/ml TNFα/IFNγ | 13.22 ± 1.24 | <.0001 |
| | 25 ng/ml TNFα/IFNγ | 15.12 ± 1.48 | <.0001 |
| | 50 ng/ml TNFα/IFNγ | 14.74 ± 1.45 | <.0001 |
| | 100 ng/ml TNFα/IFNγ | 13.64 ± 1.29 | <.0001 |
| IL-6 | Vehicle | 72.86 ± 9.77 | — |
| | 10 ng/ml TNFα/IFNγ | 2012.1 ± 337.23 | 0.0001 |
| | 25 ng/ml TNFα/IFNγ | 2329.01 ± 384.78 | <.0001 |
| | 50 ng/ml TNFα/IFNγ | 2208.6 ± 370.81 | <.0001 |
| | 100 ng/ml TNFα/IFNγ | 1889.75 ± 298.39 | 0.0004 |
| IP-10 | Vehicle | 16.61 ± 1.6 | — |
| | 10 ng/ml TNFα/IFNγ | 3275.51 ± 174.48 | <.0001 |
| | 25 ng/ml TNFα/IFNγ | 3243.28 ± 178.4.1 | <.0001 |
| | 50 ng/ml TNFα/IFNγ | 3209.56 ± 211.43 | <.0001 |
| | 100 ng/ml TNFα/IFNγ | 2978.45 ± 167.27 | <.0001 |
| MIP1α | Vehicle | 7.47 ± 1.13 | — |
| | 10 ng/ml TNFα/IFNγ | 525.75 ± 87.5 | <.0001 |
| | 25 ng/ml TNFα/IFNγ | 546.69 ± 92.35 | <.0001 |
| | 50 ng/ml TNFα/IFNγ | 531.55 ± 91.88 | <.0001 |
| | 100 ng/ml TNFα/IFNγ | 409.14 ± 60.62 | 0.0012 |
| RANTES | Vehicle | 11.78 ± 1.41 | — |
| | 10 ng/ml TNFα/IFNγ | 126.13 ± 5.15 | <.0001 |
| | 25 ng/ml TNFα/IFNγ | 127.73 ± 2.8 | <.0001 |
| | 50 ng/ml TNFα/IFNγ | 119.95 ± 4.67 | <.0001 |
| | 100 ng/ml TNFα/IFNγ | 103.48 ± 7.09 | <.0001 |

^{a}Data are presented as the mean ± standard error (SEM)

### Example D. Janus Kinase Inhibitors Interfere with Interferon-gamma and Tumor Necrosis-alpha Mediated Inflammation in Keratinocytes

Transformed human keratinocyte (HaCaT) cells were purchased from AddexBio (Catalog #T0020001) and cultured as outlined in Example C. Four compounds A-D (A: ruxolitinib, B: itacitinib ({1-{1-[3-fluoro-2-(trifluoromethyl)isonicotinoyl]piperidin-4-yl}-3 [4-(7H-pyrrolo[2,3-d]pyrimidin-4-yl)-1H-pyrazol-1-yl]azetidin-3-yl}acetonitrile), C: 4-[3-(Cyanomethyl)-3-(3 ‘,5’-dimethyl-1H,1′H-4,4′-bipyrazol-1-yl)azetidin-1-yl]-2,5-difluoro-N-[(1S)-2,2,2-trifluoro-1-methylethyl]benzamide, D: ((2R,5S)-5-{2-[(1R)-1-Hydroxyethyl]-1H-imidazo[4,5-d]thieno[3,2-b]pyridin-1-yl}tetrahydro-2H-pyran-2-yl)acetonitrile) were reconstituted in DMSO then each compound was serial diluted with cell culture media to 400 nM, 200 nM, 100 nM, and 50 nM concentrations. After 48 hours, cell culture media was removed from 24 well plates and replaced with 250 uL of media containing serial diluted drug, then incubated for 15 minutes at 37° C./5% CO₂. After drug incubation, 250 uL of combinatory stimulation containing Recombinant Human Interferon gamma (R&D Systems, Catalog #285-IF-100) and Recombinant Human Tumor Necrosis Factor alpha (R&D Systems, Catalog #210-TA-020) was added to plates. The final concentration of Recombinant Human Interferon gamma and Recombinant Human Tumor Necrosis Factor alpha was 25 ng/ml of each cytokine. Cytokine stimulation added to wells containing drug brought the final concentrations for each drug treatment to 25 nM, 50 nM, 100 nM, and 200 nM. Treated plates were mixed by gentle agitation for 30 seconds then incubated for 24 hours at 37° C./5% CO₂. At the end of the 24 hour incubation media was immediately removed from each plate.

RNA was isolated from HaCaT cells using the QuantiGene Plex Assay reagents and protocols (Affymetrix, Catalog #QGP-232-M18042302) according to the manufacturer's guidelines. Cells were washed with 1x DPBS then lysed by incubation with provided QuantiGene lysis buffer for 30 minutes at 50-55° C. Cell lysates were incubated for 18-24 hours at 55° C. with capture beads and probe set designed to specifically hybridize to mRNA from targets of interest. Genes included housekeeping genes (eg. HPRT1, GAPDH) used for the normalization of the results. After the 18-24 hour incubation signal was amplified utilizing branched DNA methodologies, according to the manufacturer's procedures (Affymetrix, Catalog #QGP-232-M18042302). After hybridization and wash steps assay plate was read on the Luminex 200 and data were expressed as Net Median Fluorescence Intensity. Data was then normalized to the Net Median Fluorescence Intensity of the housekeeping gene HPRT1 (Table 4).

TABLE 4
Normalized Expression of Target Genes in Human Keratinocyte cells Stimulated
with TNFα and IFNγ in the Presence/Absence of JAK Inhibitors

| | | | Drug | | Compound A | | Compound B | | Compound C |
|---|---|---|---|---|---|---|---|---|---|
| Gene | Stimulation^{a} | | Concentration | | MFI^{b} | p-value^{c} | MFI^{b} | p-value^{c} | MFI^{b} |
| JAK1 | — | | — | | 183.21 ± 7.55 | | | | |
| | 25 | ng/mL | — | | 213.93 ± 5.55^{£} | | | | |
| | — | | 200 | nM | 159.13 ± 7.08 | — | 171.53 ± 9.49 | — | 177.67 ± 11.84 |
| | 25 | ng/mL | 25 | nM | 206.18 ± 7.99 | 0.894 | 216.23 ± 6.41 | 0.9993 | 206.2.9 ± 6.84 |
| | 25 | ng/mL | 50 | nM | 195.48 ± 9.54 | 0.2925 | 210.42 ± 10.89 | 0.9965 | 194.2 ± 8.24 |
| | 25 | ng/mL | 100 | nM | 186.97 ± 7.49 | 0.0621 | 205.03 ± 11.49 | 0.9026 | 193.28 ± 4.55 |
| | 25 | ng/mL | 200 | nM | 180.99 ± 8.58 | 0.0191 | 195.97 ± 10.45 | 0.4597 | 182.86 ± 4.07 |
| JAK2 | — | | — | | 25.35 ± 0.95 | | | | |
| | 25 | ng/mL | — | | 126.63 ± 4.89^{¥} | | | | |
| | — | | 200 | nM | 23.67 ± 0.92 | — | 25.21 ± 1.12 | — | 25.25 ± 1.04 |
| | 25 | ng/mL | 25 | nM | 89.4 ± 2.21 | <.0001 | 109.39 ± 2.8 | 0.0021 | 114.94 ± 2.16 |
| | 25 | ng/mL | 50 | nM | 69.7 ± 1.78 | <.0001 | 101 ± 2.26 | <.0001 | 107.16 ± 2.86 |
| | 25 | ng/mL | 100 | nM | 54.4 ± 1.8 | <.0001 | 94.5 ± 2.65 | <.0001 | 95.51 ± 3.13 |
| | 25 | ng/mL | 200 | nM | 40.25 ± 1.3 | <.0001 | 89.16 ± 3.43 | <.0001 | 91.17 ± 2.15 |
| JAK3 | — | | — | | 0.66 ± 0.14 | | | | |
| | 25 | ng/mL | — | | 0.52 ± 0.16 | | | | |
| | — | | 200 | nM | 0.53 ± 0.09 | — | 0.63 ± 0.10 | — | 0.68 ± 0.17 |
| | 25 | ng/mL | 25 | nM | 0.81 ± 0.15 | 0.5284 | 0.84 ± 0.12 | 0.3247 | 1.02 ± 0.19 |
| | 25 | ng/mL | 50 | nM | 1.01 ± 0.23 | 0.1284 | 0.83 ± 0.15 | 0.3497 | 0.99 ± 0.16 |
| | 25 | ng/mL | 100 | nM | 0.84 ± 0.13 | 0.4473 | 0.92 ± 0.13 | 0.1608 | 0.99 ± 0.15 |
| | 25 | ng/mL | 200 | nM | 0.68 ± 0.13 | 0.9133 | 0.79 ± 0.15 | 0.4876 | 0.85 ± 0.15 |
| TYK2 | — | | — | | 217.40 ± 8.13 | | | | |
| | 25 | ng/mL | — | | 296.98 ± 6.92^{¥} | | | | |
| | — | | 200 | nM | 205.57 ± 10.87 | — | 217.28 ± 10.09 | — | 217.28 ± 14.28 |
| | 25 | ng/mL | 25 | nM | 298.27 ± 10.83 | >0.999 | 292.92 ± 7.99 | 0.9929 | 283.97 ± 8.59 |
| | 25 | ng/mL | 50 | nM | 287.93 ± 16.28 | 0.9305 | 287.31 ± 11.08 | 0.8603 | 273.68 ± 7.44 |
| | 25 | ng/mL | 100 | nM | 260.21 ± 7.05 | 0.0546 | 284.15 ± 9.62 | 0.7043 | 266 ± 6.82 |
| | 25 | ng/mL | 200 | nM | 264.75 ± 8.44 | 0.1204 | 277.52 ± 8.67 | 0.3578 | 263.49 ± 5.05 |
| STAT1 | — | | — | | 545.83 ± 15.37 | | | | |
| | 25 | ng/mL | — | | 3106.13 ± 217.15^{¥} | | | | |
| | — | | 200 | nM | 526.90 ± 13.46 | — | 535.07 ± 22.13 | — | 554.39 ± 11.80 |
| | 25 | ng/mL | 25 | nM | 2907.12 ± 206.85 | 0.8632 | 2868.69 ± 202.69 | 0.7833 | 3111.17 ± 182.20 |
| | 25 | ng/mL | 50 | nM | 2902.82 ± 173.71 | 0.8544 | 2862.58 ± 163.98 | 0.7685 | 3058.64 ± 154.86 |
| | 25 | ng/mL | 100 | nM | 2712.93 ± 182.91 | 0.3789 | 2790.32 ± 176.4 | 0.5807 | 3035.33 ± 122.14 |
| | 25 | ng/mL | 200 | nM | 2475.58 ± 134.64 | 0.0734 | 2857.2 ± 174.57 | 0.7553 | 2984.14 ± 163.4 |
| STAT3 | — | | — | | 751.20 ± 14.97 | | | | |
| | 25 | ng/mL | — | | 1608.39 ± 70.09^{¥} | | | | |
| | — | | 200 | nM | 728.97 ± 20.48 | — | 732.19 ± 23.03 | — | 746.17 ± 16.73 |
| | 25 | ng/mL | 25 | nM | 1434.08 ± 43.26 | 0.074 | 1466.73 ± 66.75 | 0.3206 | 1557.84 ± 58.15 |
| | 25 | ng/mL | 50 | nM | 1301.55 ± 51.7 | 0.0005 | 1437.28 ± 60.69 | 0.1762 | 1519.61 ± 69.92 |
| | 25 | ng/mL | 100 | nM | 1150.46 ± 52.66 | <.0001 | 1373.34 ± 55.51 | 0.0352 | 1457.2.4 ± 54.48 |
| | 25 | ng/mL | 200 | nM | 1082.84 ± 39.32 | <.0001 | 1400.77 ± 58.44 | 0.0738 | 1483.1 ± 51.73 |
| STAT4 | — | | — | | 4.52 ± 0.64 | | | | |
| | 25 | ng/mL | — | | 6.19 ± 0.53^{€} | | | | |
| | — | | 200 | nM | 3.75 ± 0.33 | — | 4.01 ± 0.45 | — | 4.28 ± 0.61 |
| | 25 | ng/mL | 25 | nM | 6.15 ± 10.47 | >0.999 | 6.00 ± 0.46 | 0.9967 | 5.65 ± 0.44 |
| | 2S | ng/mL | 50 | nM | 5.57 ± 0.53 | 0.7712 | 6.22 ± 0.42 | >0.999 | 5.41 ± 0.33 |
| | 25 | ng/mL | 100 | nM | 5.63 ± 0.39 | 0.8269 | 6.21 ± 0.48 | >0.999 | 5.32 ± 0.46 |
| | 25 | ng/mL | 200 | nM | 5.25 ± 0.45 | 0.4653 | 6.27 ± 0.56 | 0.9999 | 5.04 ± 0.36 |
| STAT5A | — | | — | | 2.17 ± 10.54 | | | | |
| | 25 | ng/mL | — | | 26.41 ± 2.26^{¥} | | | | |
| | — | | 200 | nM | 1.12 ± 0.19 | — | 1.44 ± 0.41 | — | 1.75 ± 0.44 |
| | 25 | ng/mL | 25 | nM | 19.04 ± 1.94 | 0.0111 | 23.69 ± 1.63 | 0.7471 | 22.82 ± 1.77 |
| | 25 | ng/mL | 50 | nM | 16.18 ± 1.66 | 0.0003 | 22.32 ± 2.16 | 0.4225 | 20.71 ± 1.77 |
| | 25 | ng/mL | 100 | nM | 12.94 ± 1.27 | <.0001 | 20.87 ± 2.1 | 0.1784 | 18.44 ± 1.85 |
| | 25 | ng/mL | 200 | nM | 9.48 ± 0.86 | <.0001 | 19.2 ± 1.94 | 0.0505 | 17.64 ± 1.46 |
| STATS | — | | — | | 749.34 ± 20.85 | | | | |
| | 25 | ng/mL | — | | 1045.99 ± 26.73^{¥} | | | | |
| | — | | 200 | nM | 723.56 ± 20.76 | — | 740.11 ± 34.98 | — | 762.04 ± 9.44 |
| | 25 | ng/mL | 25 | nM | 1043.96 ± 20.37 | >0.999 | 1004.82 ± 23.76 | 0.5557 | 1020.89 ± 23.57 |
| | 25 | ng/mL | 50 | nM | 1016.85 ± 25.68 | 0.8028 | 990.05 ± 21.06 | 0.2895 | 982.62 ± 14.34 |
| | 25 | ng/mL | 100 | nM | 966.76 ± 28.58 | 0.0739 | 987.64 ± 15.75 | 0.2557 | 943.66 ± 25.99 |
| | 25 | ng/mL | 200 | nM | 976.22 ± 14.93 | 0.1487 | 985.17 ± 29.31 | 0.224 | 966.51 ± 12.3 |
| IL-1α | — | | — | | 95.72 ± 5.84 | | | | |
| | 25 | ng/mL | — | | 1405.01 ± 27.93^{¥} | | | | |
| | — | | 200 | nM | 84.51 ± 7.04 | — | 85.16 ± 6.50 | — | 88.72 ± 5.90 |
| | 25 | ng/mL | 25 | nM | 1115.1 ± 18.96 | <.0001 | 1288.02 ± 20 | 0.0047 | 1370.52 ± 35.28 |
| | 25 | ng/mL | 50 | nM | 962.51 ± 23 | <.0001 | 1258.76 ± 23.63 | 0.0003 | 1308.7 ± 45.12 |
| | 25 | ng/mL | 100 | nM | 839.16 ± 21.04 | <.0001 | 1162.35 ± 23.34 | <.0001 | 1194.29 ± 12.27 |
| | 25 | ng/mL | 200 | nM | 755.65 ± 16.88 | <.0001 | 1126.94 ± 26.22 | <.0001 | 1151.31 ± 20.01 |
| IL-6 | — | | — | | 5.86 ± 0.38 | | | | |
| | 25 | ng/mL | — | | 170.83 ± 5.28^{¥} | | | | |
| | — | | 200 | nM | 4.70 ± 0.32 | — | 4.97 ± 0.36 | — | 4.98 ± 0.28 |
| | 25 | ng/mL | 25 | nM | 93.79 ± 4.03 | <.0001 | 130.24 ± 3.84 | <.0001 | 135.32 ± 3.36 |
| | 25 | ng/mL | 50 | nM | 69.7 ± 2.81 | <.0001 | 122.69 ± 4.36 | <.0001 | 128.14 ± 6.83 |
| | 25 | ng/mL | 100 | nM | 51.01 ± 1.57 | <.0001 | 111.07 ± 4.74 | <.0001 | 112.13 ± 3.37 |

| | | Drug | Compound C | Compound D | |
|---|---|---|---|---|---|
| Gene | Stimulation^{a} | Concentration | p-value^{c} | MFI^{b} | p-value^{c} |

| | | | |
|---|---|---|---|
| JAK1 | — | — | 183.21 ± 7.55 |

| 25 | ng/mL | — | | 213.93 ± 5.55^{£} | | |
|---|---|---|---|---|---|---|
| — | | 200 | nM | — | 177.97 ± 14.91 | — |
| 25 | ng/mL | 25 | nM | 0.7834 | 200.4 ± 9.84 | 0.5654 |
| 25 | ng/mL | 50 | nM | 0.0852 | 210.52 ± 7.73 | 0.9942 |
| 25 | ng/mL | 100 | nM | 0.0669 | 200.25 ± 8.15 | 0.5562 |
| 25 | ng/mL | 200 | nM | 0.0026 | 190.53 ± 7.68 | 0.1286 |

| | | | |
|---|---|---|---|
| JAK2 | — | — | 25.35 ± 0.95 |

| 25 | ng/mL | — | | 126.63 ± 4.89^{¥} | | |
|---|---|---|---|---|---|---|
| — | | 200 | nM | — | 25.67 ± 1.03 | — |
| 25 | ng/mL | 25 | nM | 0.0419 | 108.89 ± 3.25 | 0.0165 |
| 25 | ng/mL | 50 | nM | 0.0003 | 106.83 ± 5.94 | 0.0063 |
| 25 | ng/mL | 100 | nM | <.0001 | 102.64 ± 3.52 | 0.0007 |
| 25 | ng/mL | 200 | nM | <.0001 | 92.21 ± 2.9 | <.0001 |

| | | | |
|---|---|---|---|
| JAK3 | — | — | 0.66 ± 0.14 |

| 25 | ng/mL | — | | 0.52 ± 0.16 | | |
|---|---|---|---|---|---|---|
| — | | 200 | nM | — | 0.71 ± 0.15 | — |
| 25 | ng/mL | 25 | nM | 0.1022 | 0.97 ± 0.18 | 0.2187 |
| 25 | ng/mL | 50 | nM | 0.1493 | 0.99 ± 0.20 | 0.1854 |
| 25 | ng/mL | 100 | nM | 0.1491 | 1.01 ± 0.17 | 0.1531 |
| 25 | ng/mL | 200 | nM | 0.4323 | 0.86 ± 0.16 | 0.4442 |

| | | | |
|---|---|---|---|
| TYK2 | — | — | 217.40 ± 8.13 |

| 25 | ng/mL | — | | 296.98 ± 6.92^{¥} | | |
|---|---|---|---|---|---|---|
| — | | 200 | nM | — | 220.78 ± 12.01 | — |
| 25 | ng/mL | 25 | nM | 0.5015 | 283.93 ± 8.16 | 0.7981 |
| 25 | ng/mL | 50 | nM | 0.0823 | 307.36 ± 14.87 | 0.8958 |
| 25 | ng/mL | 100 | nM | 0.0123 | 280.63 ± 10.46 | 0.65 |
| 25 | ng/mL | 200 | nM | 0.0061 | 283.28 ± 10.88 | 0.7707 |

| | | | |
|---|---|---|---|
| STAT1 | — | — | 545.83 ± 15.37 |

| 25 | ng/mL | — | | 3106.13 ± 217.15^{¥} | | |
|---|---|---|---|---|---|---|
| — | | 200 | nM | — | 554.64 ± 11.36 | — |
| 25 | ng/mL | 25 | nM | >0.9999 | 3164.74 ± 242.35 | 0.9986 |
| 25 | ng/mL | 50 | nM | 0.9989 | 3017.08 ± 167.96 | 0.9928 |
| 25 | ng/mL | 100 | nM | 0.995 | 2999.87 ± 197.86 | 0.9862 |
| 25 | ng/mL | 200 | nM | 0.9634 | 3161.66 ± 135.8 | 0.9988 |

| | | | |
|---|---|---|---|
| STAT3 | — | — | 751.20 ± 14.97 |

| 25 | ng/mL | — | | 1608.39 ± 70.09^{¥} | | |
|---|---|---|---|---|---|---|
| — | | 200 | nM | — | 750.90 ± 27.68 | — |
| 25 | ng/mL | 25 | nM | 0.9399 | 1572.76 ± 65.5 | 0.988 |
| 25 | ng/mL | 50 | nM | 0.7044 | 1543.4 ± 58.65 | 0.9042 |
| 25 | ng/mL | 100 | nM | 0.26 | 1549.17 ± 89.41 | 0.9288 |
| 25 | ng/mL | 200 | nM | 0.4201 | 1570.19 ± 51.51 | 0.9845 |

| | | | |
|---|---|---|---|
| STAT4 | — | — | 4.52 ± 0.64 |

| 25 | ng/mL | — | | 6.19 ± 0.53^{€} | | |
|---|---|---|---|---|---|---|
| — | | 200 | nM | — | 4.32 ± 0.53 | — |
| 25 | ng/mL | 25 | nM | 0.7981 | 5.4 ± 0.45 | 0.5462 |
| 2S | ng/mL | 50 | nM | 0.5151 | 6.1 ± 0.36 | 0.9997 |
| 25 | ng/mL | 100 | nM | 0.4157 | 5.83 ± 0.34 | 0.9448 |
| 25 | ng/mL | 200 | nM | 0.1833 | 5.42 ± 0.52 | 0.5691 |

| | | | |
|---|---|---|---|
| STAT5A | — | — | 2.17 ± 10.54 |

| 25 | ng/mL | — | | 26.41 ± 2.26^{¥} | | |
|---|---|---|---|---|---|---|
| — | | 200 | nM | — | 1.99 ± 0.51 | — |
| 25 | ng/mL | 25 | nM | 0.4520 | 20.12 ± 1.29 | 0.0428 |
| 25 | ng/mL | 50 | nM | 0.1117 | 22.69 ± 1.71 | 0.3629 |
| 25 | ng/mL | 100 | nM | 0.0138 | 19.54 ± 1.34 | 0.0233 |
| 25 | ng/mL | 200 | nM | 0.0059 | 18.33 ± 1.83 | 0.0059 |

| | | | |
|---|---|---|---|
| STATS | — | — | 749.34 ± 20.85 |

| 25 | ng/mL | — | | 1045.99 ± 26.73^{¥} | | |
|---|---|---|---|---|---|---|
| — | | 200 | nM | — | 777.03 ± 29.31 | — |
| 25 | ng/mL | 25 | nM | 0.8238 | 1042.76 ± 29.23 | >0.999 |
| 25 | ng/mL | 50 | nM | 0.1389 | 1046.46 ± 29.12 | >0.999 |
| 25 | ng/mL | 100 | nM | 0.0059 | 985.1 ± 39.79 | 0.3955 |
| 25 | ng/mL | 200 | nM | 0.0429 | 1013.25 ± 17.15 | 0.8453 |

| | | | |
|---|---|---|---|
| IL-1α | — | — | 95.72 ± 5.84 |

| 25 | ng/mL | — | | 1405.01 ± 27.93^{¥} | | |
|---|---|---|---|---|---|---|
| — | | 200 | nM | — | 92.67 ± 5.54 | — |
| 25 | ng/mL | 25 | nM | 0.8379 | 1269.66 ± 50.59 | 0.0744 |
| 25 | ng/mL | 50 | nM | 0.0995 | 1336.95 ± 50.97 | 0.5871 |
| 25 | ng/mL | 100 | nM | <.0001 | 1244.96 ± 41.03 | 0.0264 |
| 25 | ng/mL | 200 | nM | <.0001 | 1163.14 ± 26.71 | 0.0004 |

| | | | |
|---|---|---|---|
| IL-6 | — | — | 5.86 ± 0.38 |

| 25 | ng/mL | — | | 170.83 ± 5.28^{¥} | | |
|---|---|---|---|---|---|---|
| — | | 200 | nM | — | 5.15 ± 0.31 | — |
| 25 | ng/mL | 25 | nM | <.0001 | 132.28 ± 7.41 | <.0001 |
| 25 | ng/mL | 50 | nM | <.0001 | 137.61 ± 5.87 | 0.0006 |
| 25 | ng/mL | 100 | nM | <.0001 | 122.46 ± 5.35 | <.0001 |

^{a}Stimulation with TNFα (25 ng/mL) and IFNγ (25 ng/mL)
^{b}Data is presented as mean ± standard error
^{c}Significant differences compared back to stimulation with TNFα and IFNγ alone
^{¥}Indicates significant difference of p < 0.0001 from vehicle (no stimulation and no drug concentration) alone
^{€}Indicates significant difference of p < 0.1 from vehicle

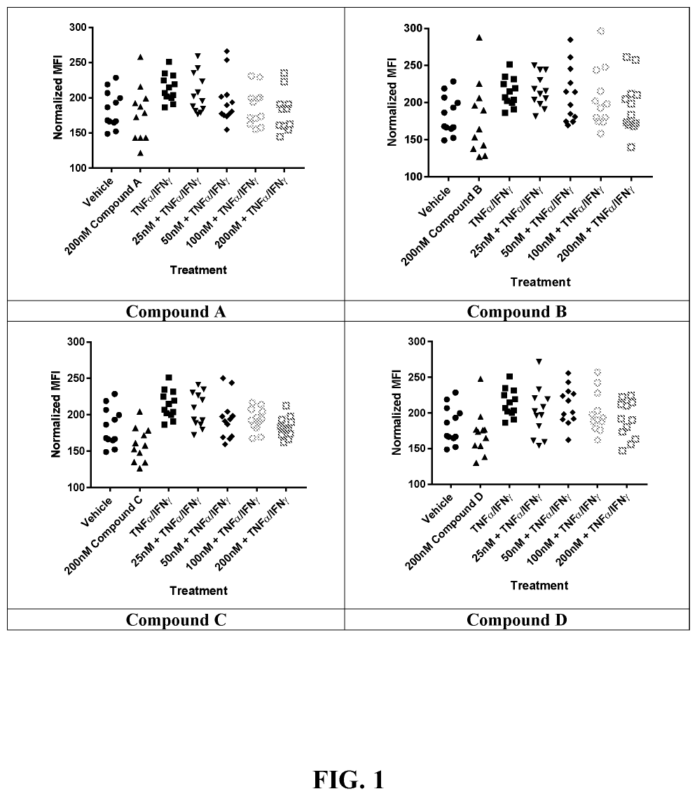

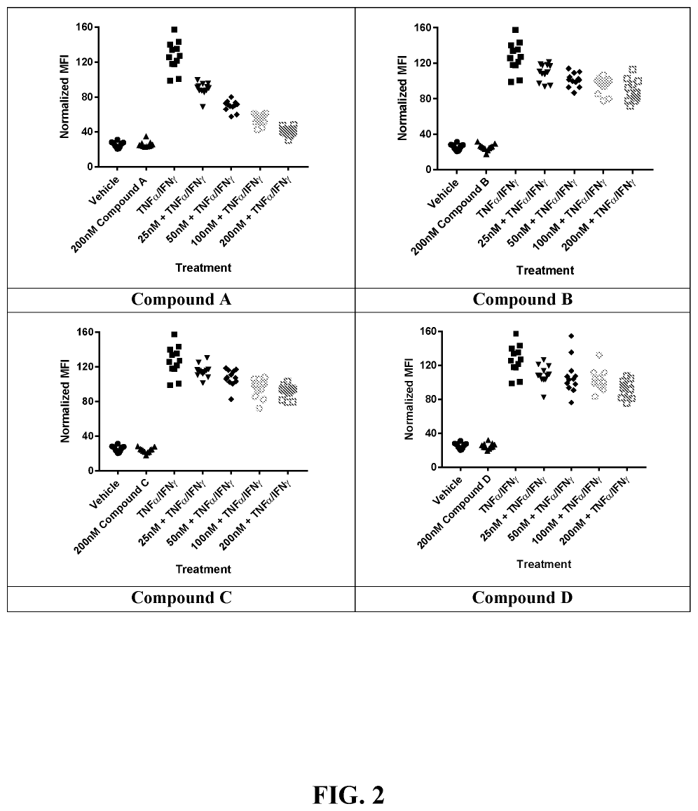

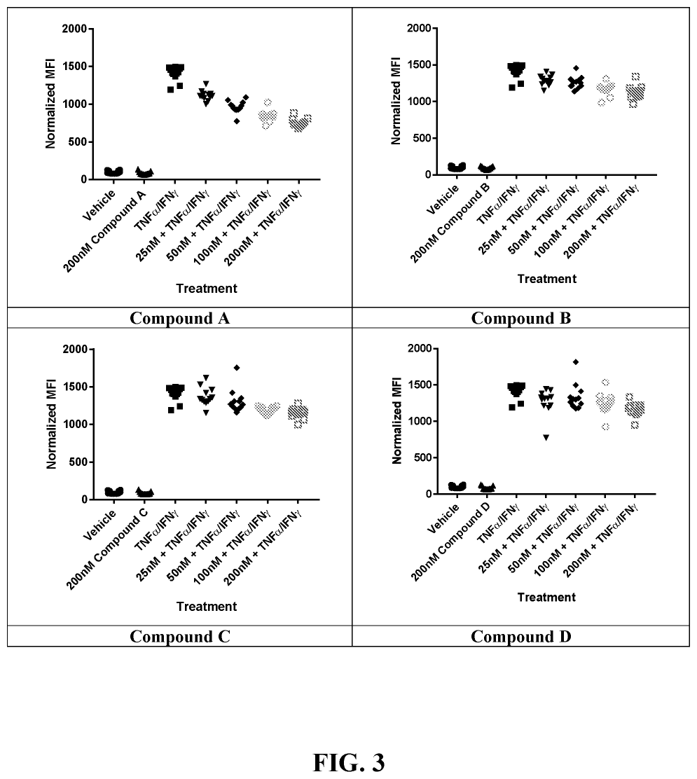

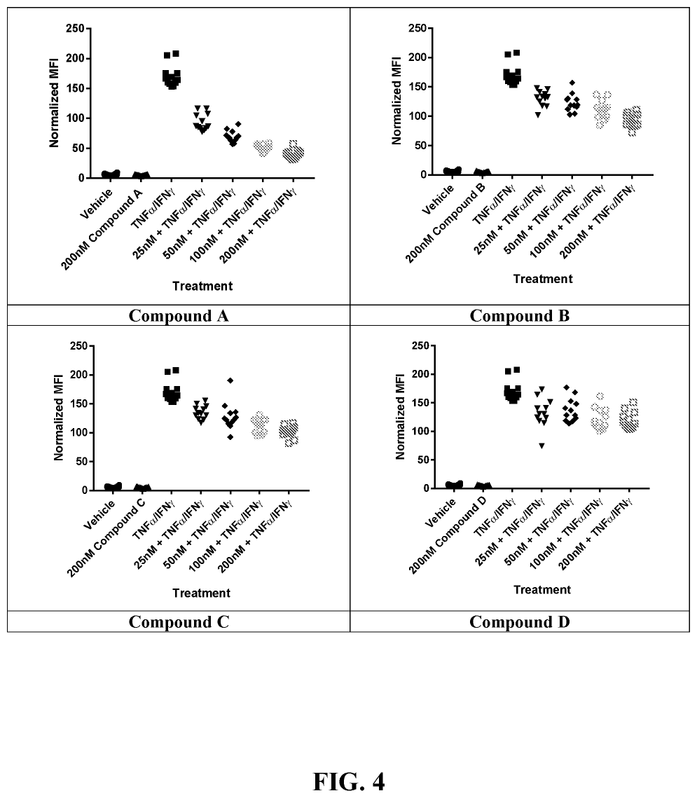

FIGS. 1-4 illustrate the individual gene expression values (MFI) for JAK1, JAK2, IL-1α, and IL-6, respectively, for each experimental replicate in keratinocytes simulated with TNFα and IFN-γ in the presence/absence of JAK inhibitors.

Target proteins of interest in the media were detected and quantified using the ProCarta Multiplex Immunoassay reagents and protocols (Invitrogen, Catalog #EPX450-12171-901). Media was incubated with antibody conjugated beads designed to bind to the epitopes of specific target proteins and identify the bound protein through the bead's distinctive spectral pattern. Biotinylated detection antibodies, designed to bind to different epitopes of the same target proteins, and Streptavidin-PE are added to assay plates to quantify the amount of the target proteins. Assay plates were read on the Luminex 200 and data were expressed as Net Median Fluorescence Intensity. The net median florescence values for the antigen standard curve, prepared according to the manufacturer's procedures (Invitrogen, Catalog #EPX450-12171-901) was plotted against the expected concentrations for each standard. The concentration of each protein was extrapolated from the antigen standard curve and concentrations were expressed as pg/mL (Table 5).

TABLE 5
Concentrations of Inflammatory Mediators Produced by Human Keratinocyte cells
Stimulated with TNFα and IFNγ in the Presence/Absence of JAK Inhibitors

| | | | Drug | | Compound A | | Compound B | | Compound C |
|---|---|---|---|---|---|---|---|---|---|
| Protein | Stimulation^{a} | | Concentration | | pg/mL^{b} | p-value^{c} | pg/mL^{b} | p-value^{c} | pg/mL^{b} |
| IL-1α | — | | — | | 0.29 ± 0.03 | | | | |
| | 25 | ng/mL | — | | 7.82 ± 0.18^{¥} | | | | |
| | — | | 200 | nM | 0.26 ± 0.05 | — | 0.29 ± 0.03 | — | 0.30 ± 0.05 |
| | 25 | ng/mL | 25 | nM | 5.93 ± 0.29 | <.0001 | 7.34 ± 0.31 | 0.7043 | 7.74 ± 0.36 |
| | 25 | ng/mL | 50 | nM | 4.9 ± 0.3 | <.0001 | 7.06 ± 0.37 | 0.3281 | 7.01 ± 0.39 |
| | 25 | ng/mL | 100 | nM | 4.12 ± 0.26 | <.0001 | 7 ± 0.41 | 0.2631 | 7.27 ± 0.47 |
| | 25 | ng/mL | 200 | nM | 3.45 ± 0.23 | <.0001 | 6.16 ± 0.35 | 0.0034 | 6.45 ± 0.38 |
| IL-6 | — | | — | | 30.57 ± 2.89 | | | | |
| | 25 | ng/mL | — | | 862.33 ± 17.95^{¥} | | | | |
| | — | | 200 | nM | 26.86 ± 2.62 | — | 28.49 ± 2.89 | — | 28.79 ± 2.91 |
| | 25 | ng/mL | 25 | nM | 594.5 ± 25.17 | <.0001 | 749.64 ± 32.94 | 0.0158 | 774.87 ± 31.09 |
| | 25 | ng/mL | 50 | nM | 446.35 ± 19.73 | <.0001 | 674.21 ± 27.15 | <.0001 | 710.89 ± 36.7 |
| | 25 | ng/mL | 100 | nM | 362.14 ± 18.73 | <.0001 | 643.8 ± 27.14 | <.0001 | 690.4 ± 35.25 |
| | 25 | ng/mL | 200 | nM | 295.21 ± 15.22 | <.0001 | 568.73 ± 24.74 | <.0001 | 621.79 ± 33.44 |
| IP-10/ | — | | — | | 20.14 ± 0.36 | | | | |
| CXCL10 | 25 | ng/mL | — | | 3935.46 ± 375.68^{¥} | | | | |
| | — | | 200 | nM | 19.75 ± 0.42 | — | 19.83 ± 0.40 | — | 20.23 ± 0.48 |
| | 25 | ng/mL | 25 | nM | 3497.56 ± 194.81 | 0.6232 | 4068.98 ± 507.12 | 0.9982 | 3999.39 ± 370.53 |
| | 25 | ng/mL | 50 | nM | 3599.04 ± 402.58 | 0.7995 | 3872.74 ± 295.01 | 0.9999 | 3665.2 ± 277.11 |
| | 25 | ng/mL | 100 | nM | 3158.24 ± 189.25 | 0.1574 | 4050.7 ± 471.31 | 0.999 | 3860.41 ± 323.05 |
| | 25 | ng/mL | 200 | nM | 2662.18 ± 89.27 | 0.0059 | 4071.78 ± 411.22 | 0.9979 | 3835.78 ± 304.58 |
| MIP1α | — | | — | | 3.14 ± 0.24 | | | | |
| | 25 | ng/mL | — | | 105.63 ± 3.74^{¥} | | | | |
| | — | | 200 | nM | 2.63 ± 0.35 | — | 2.75 ± 0.26 | — | 2.90 ± 0.21 |
| | 25 | ng/mL | 25 | nM | 82.56 ± 3.1 | <.0001 | 103.81 ± 3.29 | 0.9925 | 101.71 ± 3.84 |
| | 25 | ng/mL | 50 | nM | 70.57 ± 3.32 | <.0001 | 100.64 ± 4.66 | 0.7866 | 104.54 ± 6.56 |
| | 25 | ng/mL | 100 | nM | 50.91 ± 1.6 | <.0001 | 91.52 ± 5.05 | 0.0532 | 96.4 ± 4.18 |
| | 25 | ng/mL | 200 | nM | 40.36 ± 0.88 | <.0001 | 83.1 ± 2.77 | 0.0007 | 98.72 ± 3.87 |
| RANTES | — | | — | | 9.56 ± 0.56 | | | | |
| | 25 | ng/mL | — | | 230.17 ± 9.43^{¥} | | | | |
| | — | | 200 | nM | 10.17 ± 0.54 | — | 8.42 ± 0.51 | — | 8.61 ± 0.52 |
| | 25 | ng/mL | 25 | nM | 192 ± 12.74 | 0.0311 | 203.77 ± 12.55 | 0.4195 | 216.88 ± 13.45 |
| | 25 | ng/mL | 50 | nM | 165.12 ± 11.76 | 0.0001 | 198.35 ± 15.1 | 0.262 | 201.93 ± 15.44 |
| | 25 | ng/mL | 100 | nM | 136.24 ± 7.8 | <.0001 | 194.21 ± 12.67 | 0.1736 | 207.79 ± 17.38 |
| | 25 | ng/mL | 200 | nM | 111.94 ± 6.48 | <.0001 | 183.18 ± 13.92 | 0.0416 | 189.62 ± 13.78 |

| | | Drug | Compound C | Compound D | |
|---|---|---|---|---|---|
| Protein | Stimulation^{a} | Concentration | p-value^{c} | pg/mL^{b} | p-value^{c} |

| | | | |
|---|---|---|---|
| IL-1α | — | — | 0.29 ± 0.03 |

| 25 | ng/mL | — | | 7.82 ± 0.18^{¥} | | |
|---|---|---|---|---|---|---|
| — | | 200 | nM | — | 0.31 ± 0.04 | — |
| 25 | ng/mL | 25 | nM | 0.9994 | 6.8 ± 0.39 | 0.1498 |
| 25 | ng/mL | 50 | nM | 0.3537 | 6.76 ± 0.4 | 0.1249 |
| 25 | ng/mL | 100 | nM | 0.6747 | 6.92 ± 0.4 | 0.2281 |
| 25 | ng/mL | 200 | nM | 0.0358 | 6.3 ± 0.35 | 0.0121 |

| | | | |
|---|---|---|---|
| IL-6 | — | — | 30.57 ± 2.89 |

| 25 | ng/mL | — | | 862.33 ± 17.95^{¥} | | |
|---|---|---|---|---|---|---|
| — | | 200 | nM | — | 28.84 ± 1.89 | — |
| 25 | ng/mL | 25 | nM | 0.1794 | 743.07 ± 36.3 | 0.0476 |
| 25 | ng/mL | 50 | nM | 0.006 | 698.04 ± 29.79 | 0.0037 |
| 25 | ng/mL | 100 | nM | 0.0016 | 703.99 ± 42.22 | 0.0054 |
| 25 | ng/mL | 200 | nM | <.0001 | 646.2 ± 32.46 | <.0001 |

| IP-10/ | — | | — | 20.14 ± 0.36 |
|---|---|---|---|---|
| CXCL10 | 25 | ng/mL | — | 3935.46 ± 375.68^{¥} |

| — | | 200 | nM | — | 20.39 ± 0.57 | — |
|---|---|---|---|---|---|---|
| 25 | ng/mL | 25 | nM | 0.9998 | 3903.67 ± 366.97 | >0.999 |
| 25 | ng/mL | 50 | nM | 0.9431 | 3998.62 ± 456.34 | 0.9999 |
| 25 | ng/mL | 100 | nM | 0.9995 | 4100.26 ± 502.48 | 0.9978 |
| 25 | ng/mL | 200 | nM | 0.9984 | 4407.56 ± 645.63 | 0.8945 |

| | | | |
|---|---|---|---|
| MIP1α | — | — | 3.14 ± 0.24 |

| 25 | ng/mL | — | | 105.63 ± 3.74^{¥} | | |
|---|---|---|---|---|---|---|
| — | | 200 | nM | — | 3.11 ± 0.28 | — |
| 25 | ng/mL | 25 | nM | 0.931 | 102.06 ± 4.18 | 0.9303 |
| 25 | ng/mL | 50 | nM | 0.9994 | 96.35 ± 3.57 | 0.3335 |
| 25 | ng/mL | 100 | nM | 0.4229 | 96.22 ± 3.58 | 0.3215 |
| 25 | ng/mL | 200 | nM | 0.6469 | 88.49 ± 5.06 | 0.016 |

| | | | |
|---|---|---|---|
| RANTES | — | — | 9.56 ± 0.56 |

| 25 | ng/mL | — | | 230.17 ± 9.43^{¥} | | |
|---|---|---|---|---|---|---|
| — | | 200 | nM | — | 9.51 ± 0.56 | — |
| 25 | ng/mL | 25 | nM | 0.9096 | 237.57 ± 17.46 | 0.9967 |
| 25 | ng/mL | 50 | nM | 0.439 | 237.55 ± 21.78 | 0.9967 |
| 25 | ng/mL | 100 | nM | 0.6354 | 241.39 ± 22.79 | 0.9841 |
| 25 | ng/mL | 200 | nM | 0.1403 | 238.51 ± 23.12 | 0.9942 |

^{a}Stimulation with TNFα (25 ng/mL) and IFNγ (25 ng/mL)
^{b}Data is presented as mean ± standard error
^{c}Significant differences compared back to stimulation with TNFα and IFNγ alone
^{¥}Indicates significant difference of p < 0.0001 from vehicle (no stimulation and no drug concentration) alone

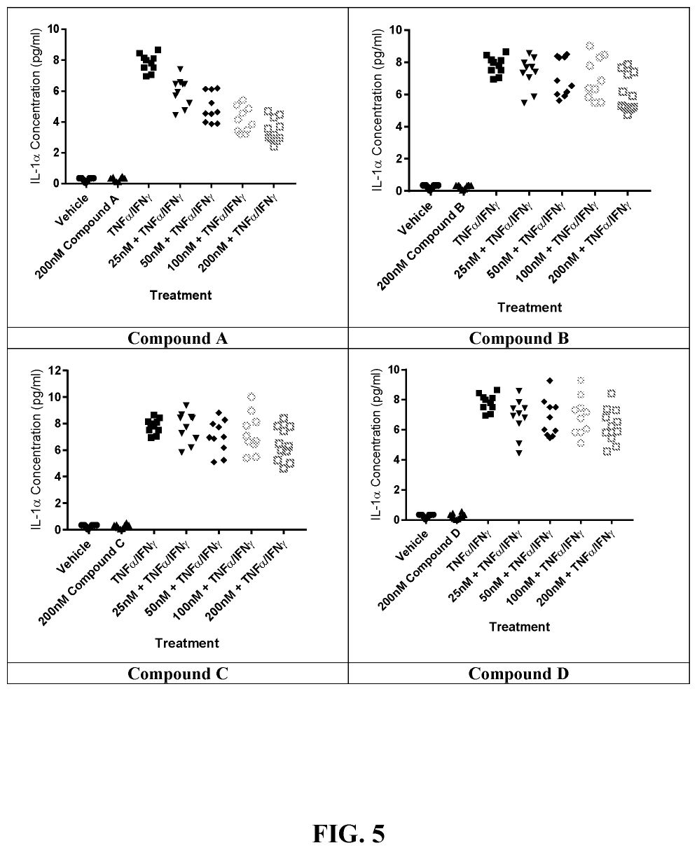

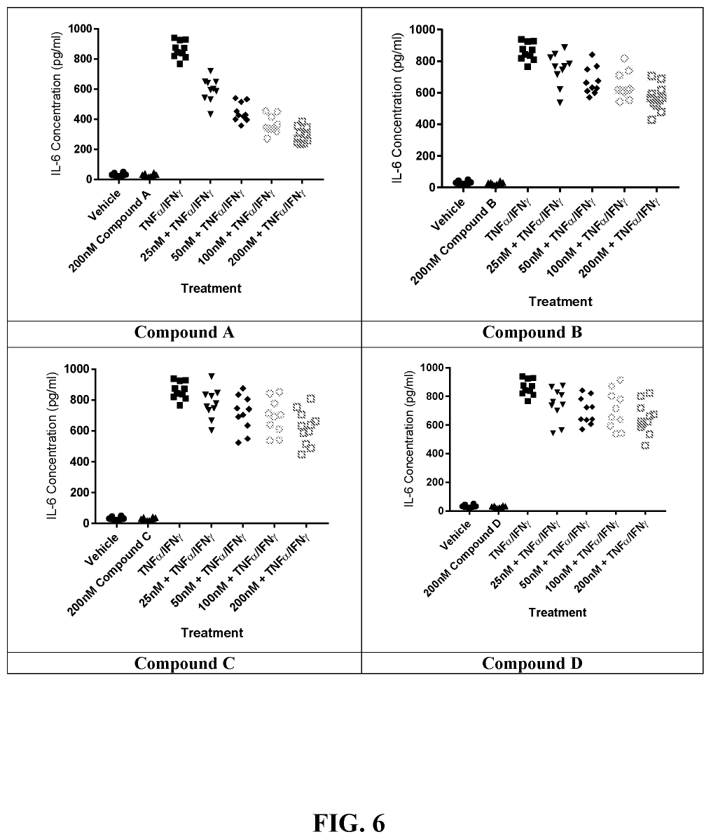

FIGS. 5 and 6 illustrate the individual protein concentrations (pg/mL) for IL-1α and IL-6, respectively, for each experimental replicate in keratinocytes simulated with TNFα and IFN-γ in the presence/absence of JAK inhibitors.

### Example E: Hidradenitis Suppurativa Skin Biopsies are characterized by Increased Janus Kinase Expression

Healthy Control skin total RNA from 3 single donors was purchased from Amsbio (Catalog #s HR101 and R1234218-50). Healthy Control skin total RNA from a pool of donors was purchased from Life Technologies Corporation (Catalog #QS0639). Hidradenitis Suppurativa Skin Biopsies (41 donors) were purchased from Discovery Life Sciences as formalin fixed paraffin embedded (FFPE) blocks from which total RNA was purified.

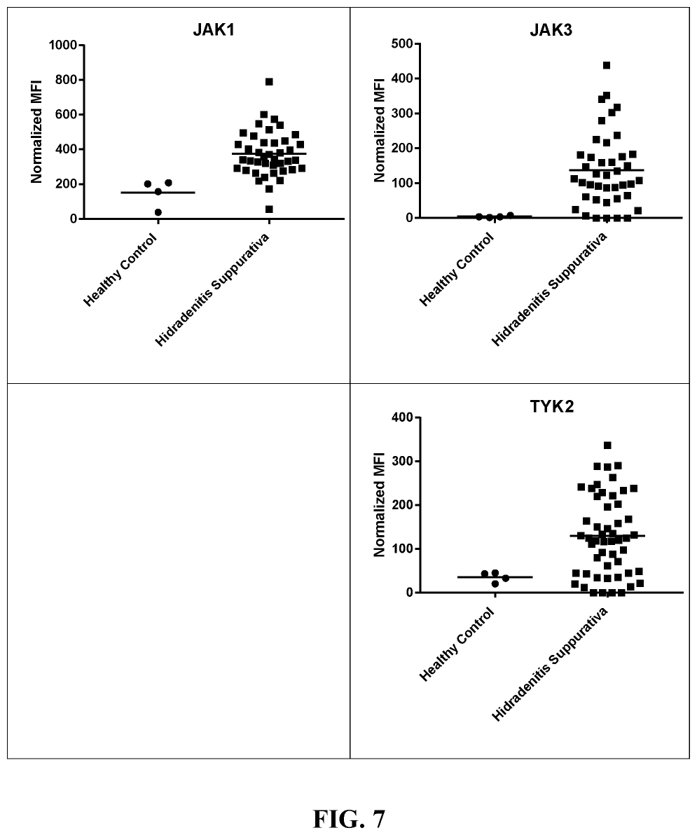

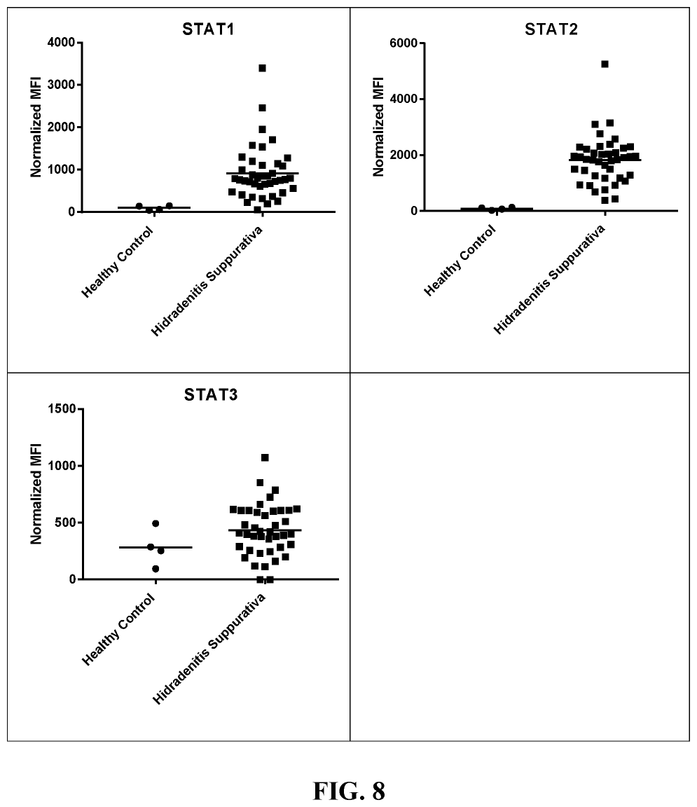

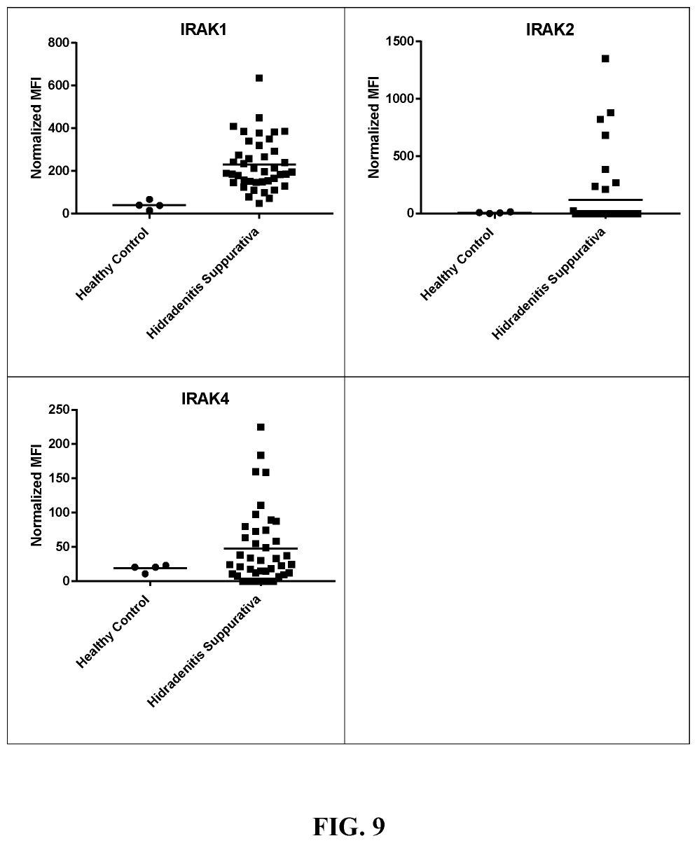

Gene expression from the Healthy Control (n=4) and Hidradenitis Suppurativa (n=41) skin total RNA samples was measured for genes outlined in Table 6 using the QuantiGene Plex Assay reagents and protocols (Life Technologies Corporation, Catalog #QGP-277-M19012402). Purified RNAs were used at the recommended assay range of 50 ng to 500 ng and were incubated overnight with capture beads designed to specifically hybridize with mRNA from selected genes (Table 6). This panel of targets included several housekeeping genes were used for normalization of the results. After overnight incubation the signal was amplified using branched DNA methodologies, according to the manufacturer's procedures (Life Technologies Corporation). The assay plate was read on a Luminex 200 and the data were expressed as Net Median Fluorescence Intensity (net MFI). Data was normalized to the geometric mean of the net MFI for the housekeeping genes ACTB and GAPDH. FIGS. 7-9 illustrate the gene expression of JAK1, JAK3, TYK2, STATI, STAT2, STAT3, IRAK1, IRAK2, and IRAK4 in the skin of healthy controls and subjects with hidradenitis suppurativa.

TABLE 6
Targeted genes

| Gene Identifier | Gene Name |
|---|---|
| JAK1 | Janus kinase 1 |
| JAK2 | Janus kinase 2 |
| JAK3 | Janus kinase 3 |
| IRAK1 | interleukin 1 receptor associated kinase 1 |
| IRAK2 | interleukin 1 receptor associated kinase 2 |
| IRAK4 | interleukin 1 receptor associated kinase 4 |
| STAT1 | signal transducer and activator of transcription 1 |
| STAT3 | signal transducer and activator of transcription 3 |
| STAT4 | signal transducer and activator of transcription 4 |
| STAT5A | signal transducer and activator of transcription 5A |
| STAR6 | signal transducer and activator of transcription 6 |
| STAT2 | signal transducer and activator of transcription 2 |
| STAT5B | signal transducer and activator of transcription 5B |
| TYK2 | tyrosine kinase 2 |
| SYK | spleen associated tyrosine kinase |
| GAPDH | glyceraldehyde-3-phosphate dehydrogenase |
| ACTB | actin beta |

Various modifications of the invention, in addition to those described herein, will be apparent to those skilled in the art from the foregoing description. Such modifications are also intended to fall within the scope of the appended claims. Each reference cited in the present application, including all patent, patent applications, and publications, is incorporated herein by reference in its entirety.

1. A method of treating hidradenitis suppurativa in a patient in need thereof, comprising administering to the patient a therapeutically effective amount of a compound which inhibits JAK1 and/or JAK2, or a pharmaceutically acceptable salt thereof, wherein the compound is:
ruxolitinib, or a pharmaceutically acceptable salt thereof, wherein one or more hydrogen atoms are replaced by deuterium atoms.

2-4. (canceled)

5. The method of claim 1, wherein the salt is ruxolitinib phosphate.

6-12. (canceled)

13. The method of claim 1, wherein the compound or salt is administered at a dosage of about 10 mg to about 60 mg on a free base basis.

14. The method of claim 1, further comprising administering an additional therapeutic agent.

15. The method of claim 14, wherein the additional therapeutic agent is an antibiotic, a retinoid, a corticosteroid, an anti-TNF-alpha agent, or an immunosuppressant.

16. The method of claim 15, wherein the antibiotic is clindamycin, doxycycline, minocycline, trimethoprim-sulfamethoxazole, erythromycin, metronidazole, rifampin, moxifloxacin, dapsone, or a combination thereof.

17. The method of claim 15, wherein the retinoid is etretinate, acitretin, or isotretinoin.

18. The method of claim 15, wherein the corticosteroid is triamcinolone, dexamethasone, fluocinolone, cortisone, prednisone, prednisolone or flumetholone.

19. The method of claim 15, wherein the anti-TNF-alpha agent is infliximab, etanercept, or adalimumab.

20. The method of claim 15, wherein the immunosuppressant is methotrexate, cyclosporin A, mycophenolate mofetil, or mycophenolate sodium.

21. The method of claim 14, wherein the additional therapeutic agent is finasteride, metformin, adapalene, or azelaic acid.

22. The method of claim 1, wherein the administering of the compound or salt is topical.

23. The method of claim 1, wherein the administering of the compound or salt is oral.

24. The method of claim 1, wherein the method results in a 10%, 20%, 30%, 40%, or 50% improvement in HiSCR (Hidradenitis Suppurativa Clinical Response).

25. The method of claim 1, wherein the method reduces inflammatory nodules resulting from hidradenitis suppurativa.

26. The method of claim 1, wherein the method reduces abscesses resulting from hidradenitis suppurativa.

27. The method of claim 1, wherein the method reduces abscesses and inflammatory nodules resulting from hidradenitis suppurativa.
